SEQUENCE LISTING (20 sequences)

SEQ ID NO: 1 - protein, 5 aa, Artificial Sequence
  Synthetic
NYGVH

SEQ ID NO: 2 - protein, 16 aa, Artificial Sequence
  Synthetic
VIWSGGNTDYNTPFTS

SEQ ID NO: 3 - protein, 11 aa, Artificial Sequence
  Synthetic
ALTYYDYEFAY

SEQ ID NO: 4 - protein, 11 aa, Artificial Sequence
  Synthetic
RASQSIGTNIH

SEQ ID NO: 5 - protein, 7 aa, Artificial Sequence
  Synthetic
YASESIS

SEQ ID NO: 6 - protein, 9 aa, Artificial Sequence
  Synthetic
QQNNNWPTT

SEQ ID NO: 7 - protein, 7 aa, Artificial Sequence
  Synthetic
SGDYYWT

SEQ ID NO: 8 - protein, 16 aa, Artificial Sequence
  Synthetic
HIYYSGNTNYNPSLKS

SEQ ID NO: 9 - protein, 9 aa, Artificial Sequence
  Synthetic
DRVTGAFDI

SEQ ID NO: 10 - protein, 11 aa, Artificial Sequence
  Synthetic
QASQDISNYLN

SEQ ID NO: 11 - protein, 7 aa, Artificial Sequence
  Synthetic
DASNLET

SEQ ID NO: 12 - protein, 9 aa, Artificial Sequence
  Synthetic
QHFDHLPLA

SEQ ID NO: 13 - protein, 9 aa, Artificial Sequence
  Synthetic
GGSISSGDY

SEQ ID NO: 14 - protein, 5 aa, Artificial Sequence
  Synthetic
YYSGS

SEQ ID NO: 15 - protein, 11 aa, Artificial Sequence
  Synthetic
VSIFGVGTFDY

SEQ ID NO: 16 - protein, 11 aa, Artificial Sequence
  Synthetic
RASQSVSSYLA

SEQ ID NO: 17 - protein, 7 aa, Artificial Sequence
  Synthetic
DASNRAT

SEQ ID NO: 18 - protein, 9 aa, Artificial Sequence
  Synthetic
HQYGSTPLT

SEQ ID NO: 19 - protein, 119 aa, Artificial Sequence
  Synthetic
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 20 - protein, 107 aa, Artificial Sequence
  Synthetic
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

(19) United States
(12) Patent Application Publication
(10) Pub. No.: US 2021/0187115 A1
(43) Pub. Date: Jun. 24, 2021

(54) Immunoconjugates Targeting EGFR

(71) Applicants: Bolt Biotherapeutics, Inc., Redwood City, CA (US); The Board of Trustees of The Leland Stanford Junior University, Stanford, CA (US)

(72) Inventors: Michael N. Alonso, Redwood City, CA (US); Edgar George Engleman, Redwood City, CA (US); David Y. Jackson, Redwood City, CA (US); Arthur Lee, Redwood City, CA (US); Shelley Erin Ackerman, Redwood City, CA (US)

(73) Assignees: Bolt Biotherapeutics, Inc., Redwood City, CA (US); The Board of Trustees of The Leland Stanford Junior University, Stanford, CA (US)

(21) Appl. No.: 17/271,896

(22) PCT Filed: Aug. 29, 2019
(86) PCT No.: PCT/US2019/048706

§ 371 Date: Feb. 26, 2021

Related U.S. Application Data
(60) Provisional application No. 62/724,550, filed on Aug. 29, 2018.

(51) Int. Cl. A61K 47/68; A61P 35/00; C07K 16/28

(52) U.S. Cl. CPC A61K 47/6803; C07K 16/2863; A61K 47/6849; A61P 35/00

(57) ABSTRACT

The invention provides an immunoconjugate of formula: (I), or pharmaceutically acceptable salt thereof, wherein subscript r is an integer from 1 to 10, subscript n is an integer from about 2 to about 50, “Adj” is an adjuvant moiety, and “Ab” is an antibody construct that has an antigen binding domain that binds EGFR. The invention also provides compositions comprising the immunoconjugate. The invention further provides methods of treating cancer with the immunoconjugate.

### CROSS-REFERENCE TO RELATED APPLICATION

This patent application claims the benefit of U.S. Provisional Patent Application No. 62/724,550, filed Aug. 29, 2018, which is incorporated by reference in its entirety herein.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 5,795 Byte ASCII (Text) file named “744859_ST25.txt,” created on Aug. 22, 2019.

### BACKGROUND OF THE INVENTION

It is now well appreciated that tumor growth necessitates the acquisition of mutations that facilitate immune evasion. Even so, tumorigenesis results in the accumulation of mutated antigens, or neoantigens, that are readily recognized by the host immune system following ex vivo stimulation. Why and how the immune system fails to recognize neoantigens are beginning to be elucidated. Groundbreaking studies by Carmi et al. (Nature, 521: 99-104 (2015)) have indicated that immune ignorance can be overcome by delivering neoantigens to activated dendritic cells via antibody-tumor immune complexes. In these studies, simultaneous delivery of tumor binding antibodies and dendritic cell adjuvants via intratumoral injections resulted in robust anti-tumor immunity. New compositions and methods for the delivery of antibodies and dendritic cell adjuvants are needed in order to reach inaccessible tumors and/or to expand treatment options for cancer patients and other subjects. The invention provides such compositions and methods.

### BRIEF SUMMARY OF THE INVENTION

The invention provides an immunoconjugate of formula:

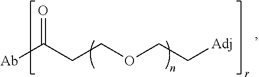

or pharmaceutically acceptable salt thereof, wherein subscript r is an integer from 1 to 10, subscript n is an integer from about 2 to about 50, “Adj” is an adjuvant moiety, and “Ab” is an antibody construct that has an antigen binding domain that binds epidermal growth factor receptor (“EGFR”).

The invention further provides an immunoconjugate of formula:

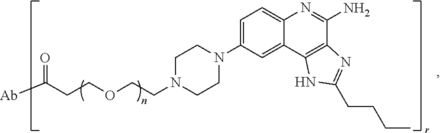

or pharmaceutically acceptable salt thereof, wherein subscript r is an integer from 1 to 10, subscript n is an integer from about 2 to about 50, and “Ab” is an antibody construct that has an antigen binding domain that binds EGFR.

The invention provides a composition comprising a plurality of immunoconjugates described herein.

The invention provides a method for treating and/or curing cancer in a subject comprising administering a therapeutically effective amount of an immunoconjugate or a composition described herein to a subject in need thereof.

### BRIEF DESCRIPTION OF THE DRAWINGS

FIG. 1 is a graph of tumor volume versus days, which illustrates the ability of the immunoconjugates of the invention to act as potent anti-tumor therapies, as exhibited by treatment of a human tumor model for colorectal cancer, COLO 205.

FIG. 2 is a graph of tumor volume versus days, which illustrates the ability of the immunoconjugates of the invention to act as potent anti-tumor therapies, as exhibited by treatment of a human tumor model for lung adenocarcinoma, HCC827.

FIG. 3 is a graph of fold change versus concentration, which shows that Immunoconjugate 1 and Immunoconjugate 2 elicit myeloid activation as indicated by CD40 upregulation.

FIG. 4 is a graph of fold change versus concentration, which shows that Immunoconjugate 1 and Immunoconjugate 2 elicit myeloid activation as indicated by CD86 upregulation.

FIG. 5 is a graph of fold change versus concentration, which shows that Immunoconjugate 1 and Immunoconjugate 2 elicit myeloid differentiation as indicated by CD16 downregulation.

FIG. 6 is a graph of fold change versus concentration, which shows that Immunoconjugate 1 and Immunoconjugate 2 elicit myeloid activation as indicated by CD123 upregulation.

### DETAILED DESCRIPTION OF THE INVENTION

### General

The invention provides an immunoconjugate of formula:

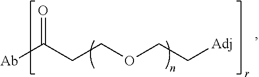

or pharmaceutically acceptable salt thereof, wherein subscript r is an integer from 1 to 10, subscript n is an integer from about 2 to about 50, “Adj” is an adjuvant moiety, and “Ab” is an antibody construct that has an antigen binding domain that binds epidermal growth factor receptor (“EGFR”).

Antibody-adjuvant immunoconjugates of the invention, comprising an antibody construct that has an antigen binding domain that binds EGFR linked to an adjuvant moiety, demonstrate superior pharmacological properties over conventional antibody conjugates. The polyethylene glycol-based linker (“PEG linker”) is the preferred linker to provide adequate purification and isolation of the immunoconjugate, maintain function of the adjuvant moiety and antibody construct, and produce ideal pharmacokinetic (“PK”) properties of the immunoconjugate. Additional embodiments and benefits of the inventive antibody-adjuvant immunoconjugates will be apparent from description herein.

### Definitions

As used herein, the term “immunoconjugate” refers to an antibody construct that is covalently bonded to an adjuvant moiety via a linker.

As used herein, the phrase “antibody construct” refers to an antibody or a fusion protein comprising (i) an antigen binding domain and (ii) an Fc domain.

As used herein, the term “antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as numerous immunoglobulin variable region genes.

An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa) connected by disulfide bonds. Each chain is composed of structural domains, which are referred to as immunoglobulin domains. These domains are classified into different categories by size and function, e.g., variable domains or regions on the light and heavy chains (V_{L} and V_{H}, respectively) and constant domains or regions on the light and heavy chains (C_{L} and C_{H}, respectively). The N-terminus of each chain defines a variable region of about 100 to 110 or more amino acids, referred to as the paratope, primarily responsible for antigen recognition, i.e., the antigen-binding site. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, IgG2, IgG3, and IgG4) in humans, named in order of their abundance in serum (i.e., IgG1 is the most abundant). Typically, the antigen-binding region of an antibody will be most critical in specificity and affinity of binding to cancer cells.

Antibodies can exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see, e.g., Fundamental Immunology (Paul, editor, 7th edition, 2012)). While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv), or those identified using phage display libraries (see, e.g., McCafferty et al., Nature, 348: 552-554 (1990)).

The term “antibody” is used in the broadest sense and specifically encompasses monoclonal antibodies (including full length monoclonal antibodies), polyclonal antibodies, multispecific antibodies (e.g., bispecific antibodies), and antibody fragments that exhibit the desired biological activity. “Antibody fragment” and all grammatical variants thereof as used herein are defined as a portion of an intact antibody comprising the antigen binding site or variable region of the intact antibody, wherein the portion is free of the constant heavy chain domains (i.e., CH2, CH3, and CH4, depending on antibody isotype) of the Fc region of the intact antibody. Examples of antibody fragments include Fab, Fab′, Fab′-SH, F(ab′)2, and Fv fragments; diabodies; camelid nanobodies (VHHs); any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules; (2) single chain polypeptides containing only one light chain variable domain, or a fragment thereof that contains the three CDRs of the light chain variable domain, without an associated heavy chain moiety; (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g., CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used herein, the term “epitope” means any antigenic determinant or epitopic determinant of an antigen to which an antibody binds (i.e., at the paratope of the antibody). Antigenic determinants usually consist of chemically active surface groupings of molecules, such as amino acids or sugar side chains, and usually have specific three dimensional structural characteristics, as well as specific charge characteristics.

As used herein, the term “adjuvant” refers to a substance capable of eliciting an immune response in a subject exposed to the adjuvant. The phrase “adjuvant moiety” refers to an adjuvant that is covalently bonded to an antibody as described herein. The adjuvant moiety can elicit the immune response while bonded to the antibody or after cleavage (e.g., enzymatic cleavage) from the antibody following administration of an immunoconjugate to the subject.

As used herein, the terms “Toll-like receptor” and “TLR” refer to any member of a family of highly-conserved mammalian proteins which recognizes pathogen-associated molecular patterns and acts as key signaling elements in innate immunity. TLR polypeptides share a characteristic structure that includes an extracellular domain that has leucine-rich repeats, a transmembrane domain, and an intracellular domain that is involved in TLR signaling.

The terms “Toll-like receptor 7” and “TLR7” refer to nucleic acids or polypeptides sharing at least 70%, 80%, 90%, 95%, 96%, 97%, 98%, 99%, or more sequence identity to a publicly-available TLR7 sequence, e.g., GenBank accession number AAZ99026 for human TLR7 polypeptide, or GenBank accession number AAK62676 for murine TLR7 polypeptide.

The terms “Toll-like receptor 8” and “TLR8” refer to nucleic acids or polypeptides sharing at least 70%, 80%, 90%, 95%, 96%, 97%, 98%, 99%, or more sequence identity to a publicly-available TLR7 sequence, e.g., GenBank accession number AAZ95441 for human TLR8 polypeptide, or GenBank accession number AAK62677 for murine TLR8 polypeptide.

A “TLR agonist” is a substance that binds, directly or indirectly, to a TLR (e.g., TLR7 and/or TLR8) to induce TLR signaling. Any detectable difference in TLR signaling can indicate that an agonist stimulates or activates a TLR. Signaling differences can be manifested, for example, as changes in the expression of target genes, in the phosphorylation of signal transduction components, in the intracellular localization of downstream elements such as nuclear factor-KB(NF-κB), in the association of certain components (such as IL-1 receptor associated kinase (IRAK)) with other proteins or intracellular structures, or in the biochemical activity of components such as kinases (such as mitogen-activated protein kinase (MAPK)).

As used herein, “Ab” refers to an antibody construct that has an antigen binding domain that binds EGFR (e.g., cetuximab (also known as ERBITUX™), panitumumab (also known as VECTIBIX™), or necitumumab (also known as PORTRAZZA™), a biosimilar thereof, or a biobetter thereof). In some embodiments, “Ab” is selected from panitumumab, cetuximab, necitumumab, STI-001, RPH-002, CMAB009, ONS-1055, MabionEGFR, HLX-05, HLX05, CT-P15, KN-005, ABP-494, AP-087, EMD72000 (also known as matuzumab), futuximab, modotuximab, tomuzotuximab (also known as CETUGEX™), imgatuzumab, MDX-214, Mab-806, JNJ-6372, ATC-EGFRBi, GC1118, SYN004, SCT200, EMD-55900, ICR-62, or HLX-07.

An embodiment of the invention provides an antibody comprising the CDR regions of the anti-EGFR antibody cetuximab. In this regard, the antibody may comprise a first variable region comprising a CDR1 comprising the amino acid sequence of SEQ ID NO: 1 (CDR1 of first variable region), a CDR2 comprising the amino acid sequence of SEQ ID NO: 2 (CDR2 of first variable region), and a CDR3 comprising the amino acid sequence of SEQ ID NO: 3 (CDR3 of first variable region), and a second variable region comprising a CDR1 comprising the amino acid sequence of SEQ ID NO: 4 (CDR1 of second variable region), a CDR2 comprising the amino acid sequence of SEQ ID NO: 5 (CDR2 of second variable region), and a CDR3 comprising the amino acid sequence of SEQ ID NO: 6 (CDR3 of second variable region). In this regard, an antibody can comprise (i) all of SEQ ID NOs: 1-3, (ii) all of SEQ ID NOs: 4-6, or (iii) all of SEQ ID NOs: 1-6. Preferably, the antibody comprises all of SEQ ID NOs: 1-6.

An embodiment of the invention provides an antibody comprising the CDR regions of the anti-EGFR antibody panitumumab. In this regard, the antibody may comprise a first variable region comprising a CDR1 comprising the amino acid sequence of SEQ ID NO: 7 (CDR1 of first variable region), a CDR2 comprising the amino acid sequence of SEQ ID NO: 8 (CDR2 of first variable region), and a CDR3 comprising the amino acid sequence of SEQ ID NO: 9 (CDR3 of first variable region), and a second variable region comprising a CDR1 comprising the amino acid sequence of SEQ ID NO: 10 (CDR1 of second variable region), a CDR2 comprising the amino acid sequence of SEQ ID NO: 11 (CDR2 of second variable region), and a CDR3 comprising the amino acid sequence of SEQ ID NO: 12 (CDR3 of second variable region). In this regard, an antibody can comprise (i) all of SEQ ID NOs: 7-9, (ii) all of SEQ ID NOs: 10-12, or (iii) all of SEQ ID NOs: 7-12. Preferably, the antibody comprises all of SEQ ID NOs: 7-12.

An embodiment of the invention provides an antibody comprising the CDR regions of the anti-EGFR antibody necitumumab. In this regard, the antibody may comprise a first variable region comprising a CDR1 comprising the amino acid sequence of SEQ ID NO: 13 (CDR1 of first variable region), a CDR2 comprising the amino acid sequence of SEQ ID NO: 14 (CDR2 of first variable region), and a CDR3 comprising the amino acid sequence of SEQ ID NO: 15 (CDR3 of first variable region), and a second variable region comprising a CDR1 comprising the amino acid sequence of SEQ ID NO: 16 (CDR1 of second variable region), a CDR2 comprising the amino acid sequence of SEQ ID NO: 17 (CDR2 of second variable region), and a CDR3 comprising the amino acid sequence of SEQ ID NO: 18 (CDR3 of second variable region). In this regard, an antibody can comprise (i) all of SEQ ID NOs: 13-15, (ii) all of SEQ ID NOs: 16-18, or (iii) all of SEQ ID NOs: 13-18. Preferably, the antibody comprises all of SEQ ID NOs: 13-18.

An embodiment of the invention provides an antibody comprising one or both variable regions of the anti-EGFR antibody cetuximab. In this regard, the first variable region may comprise SEQ ID NO: 19. The second variable region may comprise SEQ ID NO: 20. Accordingly, in an embodiment of the invention, the antibody comprises SEQ ID NO: 19, SEQ ID NO: 20, or both SEQ ID NOs: 19 and 20. Preferably, the antibody comprises both of SEQ ID NOs: 19-20.

An embodiment of the invention provides an anti-EGFR antibody (e.g., cetuximab, necitumumab, panitumumab, or a biosimilar or biobetter thereof) comprising a sequence that is at least about 70% or more, e.g., about 80%, about 90%, about 91%, about 92%, about 93%, about 94%, about 95%, about 96%, about 97%, about 98%, or about 99% identical to any of the amino acid sequences described herein (i.e., any one of SEQ ID NOs: 1-20).

As used herein, the term “biosimilar” refers to an approved antibody construct that has active properties similar to the antibody construct previously approved (e.g., cetuximab, panitumumab, or necitumumab).

As used herein, the term “biobetter” refers to an approved antibody construct that is an improvement of a previously approved antibody construct (e.g., cetuximab, panitumumab, or necitumumab). The biobetter can have one or more modifications (e.g., an altered glycan profile, or a unique epitope) over the previously approved antibody construct.

As used herein, the term “amino acid” refers to any monomeric unit that can be incorporated into a peptide, polypeptide, or protein. Amino acids include naturally-occurring α-amino acids and their stereoisomers, as well as unnatural (non-naturally occurring) amino acids and their stereoisomers. “Stereoisomers” of a given amino acid refer to isomers having the same molecular formula and intramolecular bonds but different three-dimensional arrangements of bonds and atoms (e.g., an L-amino acid and the corresponding D-amino acid). The amino acids can be glycosylated (e.g., N-linked glycans, O-linked glycans, phosphoglycans, C-linked glycans, or glypiation) or deglycosylated.

Naturally-occurring amino acids are those encoded by the genetic code, as well as those amino acids that are later modified, e.g., hydroxyproline, γ-carboxyglutamate, and O-phosphoserine. Naturally-occurring α-amino acids include, without limitation, alanine (Ala), cysteine (Cys), aspartic acid (Asp), glutamic acid (Glu), phenylalanine (Phe), glycine (Gly), histidine (His), isoleucine (Ile), arginine (Arg), lysine (Lys), leucine (Leu), methionine (Met), asparagine (Asn), proline (Pro), glutamine (Gln), serine (Ser), threonine (Thr), valine (Val), tryptophan (Trp), tyrosine (Tyr), and combinations thereof. Stereoisomers of naturally-occurring α-amino acids include, without limitation, D-alanine (D-Ala), D-cysteine (D-Cys), D-aspartic acid (D-Asp), D-glutamic acid (D-Glu), D-phenylalanine (D-Phe), D-histidine (D-His), D-isoleucine (D-Ile), D-arginine (D-Arg), D-lysine (D-Lys), D-leucine (D-Leu), D-methionine (D-Met), D-asparagine (D-Asn), D-proline (D-Pro), D-glutamine (D-Gln), D-serine (D-Ser), D-threonine (D-Thr), D-valine (D-Val), D-tryptophan (D-Trp), D-tyrosine (D-Tyr), and combinations thereof.

Unnatural (non-naturally occurring) amino acids include, without limitation, amino acid analogs, amino acid mimetics, synthetic amino acids, N-substituted glycines, and N-methyl amino acids in either the L- or D-configuration that function in a manner similar to the naturally-occurring amino acids. For example, “amino acid analogs” can be unnatural amino acids that have the same basic chemical structure as naturally-occurring amino acids (i.e., a carbon that is bonded to a hydrogen, a carboxyl group, an amino group) but have modified side-chain groups or modified peptide backbones, e.g., homoserine, norleucine, methionine sulfoxide, and methionine methyl sulfonium. “Amino acid mimetics” refer to chemical compounds that have a structure that is different from the general chemical structure of an amino acid, but that functions in a manner similar to a naturally-occurring amino acid.

Amino acids may be referred to herein by either the commonly known three letter symbols or by the one-letter symbols recommended by the IUPAC-IUB Biochemical Nomenclature Commission.

As used herein, the term “alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆, and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl can also refer to alkyl groups having up to 30 carbons atoms, such as, but not limited to, heptyl, octyl, nonyl, decyl, etc. Alkyl groups can be substituted or unsubstituted. “Substituted alkyl” groups can be substituted with one or more groups selected from halo, hydroxy, amino, oxo (═O), alkylamino, amido, acyl, nitro, cyano, and alkoxy. The term “alkylene” refers to a divalent alkyl radical.

As used herein, the term “heteroalkyl” refers to an alkyl group as described herein, wherein each of one or more carbon atoms is optionally and independently replaced with a heteroatom selected from N, O, and S. The term “heteroalkylene” refers to a divalent heteroalkyl radical.

As used herein, the term “carboalkyl” refers to a saturated or partially unsaturated, monocyclic, fused bicyclic, or bridged polycyclic ring assembly containing from 3 to 12 ring atoms, or the number of atoms indicated. Carboalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic carbocyclic rings include, for example, cyclopropyl, cyclobutyl, cyclopentyl, cyclohexyl, and cyclooctyl. Saturated bicyclic and polycyclic carbocyclic rings include, for example, norbornane, [2.2.2] bicyclooctane, decahydronaphthalene, and adamantane. Carbocyclic groups can also be partially unsaturated by having one or more double or triple bonds in the ring. Representative carbocyclic groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbornene, and norbornadiene.

Unsaturated carbocyclic groups also include aryl groups. The term “aryl” refers to an aromatic ring system having any suitable number of ring atoms and any suitable number of rings. Aryl groups can include any suitable number of ring atoms, such as, 6, 7, 8, 9, 10, 11, 12, 13, 14, 15, or 16 ring atoms, as well as from 6 to 10, 6 to 12, or 6 to 14 ring atoms. Aryl groups can be monocyclic, fused to form bicyclic or tricyclic groups, or linked by a bond to form a biaryl group. Representative aryl groups include phenyl, naphthyl, and biphenyl. Other aryl groups include benzyl, which has a methylene linking group. Some aryl groups have from 6 to 12 ring atoms, such as phenyl, naphthyl, or biphenyl. Other aryl groups have from 6 to 10 ring atoms, such as phenyl or naphthyl.

A “divalent” carboalkyl refers to a carbocyclic group having two points of attachment for covalently linking two moieties in a molecule or material. Carboalkyls can be substituted or unsubstituted. “Substituted carboalkyl” groups can be substituted with one or more groups selected from halo, hydroxy, amino, alkylamino, amido, acyl, nitro, cyano, and alkoxy.

As used herein, the term “heterocycle” refers to heterocycloalkyl groups and heteroaryl groups. “Heteroaryl,” by itself or as part of another substituent, refers to a monocyclic or fused bicyclic or tricyclic aromatic ring assembly containing 5 to 16 ring atoms, where each of from 1 to 5 of the ring atoms is a heteroatom, such as N, O, or S. Suitable heteroatoms also include, but are not limited to, B, Al, Si, and P. The heteroatoms can be oxidized to form moieties, such as, but not limited to, —S(O)— and —S(O)₂—. Heteroaryl groups can include any number of ring atoms, such as 3 to 6, 4 to 6, 5 to 6, 3 to 8, 4 to 8, 5 to 8, 6 to 8, 3 to 9, 3 to 10, 3 to 11, or 3 to 12 ring atoms. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted. “Substituted heteroaryl” groups can be substituted with one or more groups selected from halo, hydroxy, amino, oxo (═O), alkylamino, amido, acyl, nitro, cyano, and alkoxy.

Heteroaryl groups can be linked via any position on the ring. For example, pyrrole includes 1-, 2- and 3-pyrrole, pyridine includes 2-, 3- and 4-pyridine, imidazole includes 1-, 2-, 4- and 5-imidazole, pyrazole includes 1-, 3-, 4- and 5-pyrazole, triazole includes 1-, 4- and 5-triazole, tetrazole includes 1- and 5-tetrazole, pyrimidine includes 2-, 4-, 5- and 6-pyrimidine, pyridazine includes 3- and 4-pyridazine, 1,2,3-triazine includes 4- and 5-triazine, 1,2,4-triazine includes 3-, 5- and 6-triazine, 1,3,5-triazine includes 2-triazine, thiophene includes 2- and 3-thiophene, furan includes 2- and 3-furan, thiazole includes 2-, 4- and 5-thiazole, isothiazole includes 3-, 4- and 5-isothiazole, oxazole includes 2-, 4- and 5-oxazole, isoxazole includes 3-, 4- and 5-isoxazole, indole includes 1-, 2- and 3-indole, isoindole includes 1- and 2-isoindole, quinoline includes 2-, 3- and 4-quinoline, isoquinoline includes 1-, 3- and 4-isoquinoline, quinazoline includes 2- and 4-quinoazoline, cinnoline includes 3- and 4-cinnoline, benzothiophene includes 2- and 3-benzothiophene, and benzofuran includes 2- and 3-benzofuran.

“Heterocycloalkyl,” by itself or as part of another substituent, refers to a saturated ring system having from 3 to 12 ring atoms and from 1 to 4 heteroatoms of N, O, and S. Suitable heteroatoms also be include, but are not limited to, B, Al, Si, and P. The heteroatoms can be oxidized to form moieties, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring atoms. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. “Substituted heterocycloalkyl” groups can be substituted with one or more groups selected from halo, hydroxy, amino, oxo (═O), alkylamino, amido, acyl, nitro, cyano, and alkoxy.

Heterocycloalkyl groups can be linked via any position on the ring. For example, aziridine can be 1- or 2-aziridine, azetidine can be 1- or 2-azetidine, pyrrolidine can be 1-, 2- or 3-pyrrolidine, piperidine can be 1-, 2-, 3- or 4-piperidine, pyrazolidine can be 1-, 2-, 3-, or 4-pyrazolidine, imidazolidine can be 1-, 2-, 3- or 4-imidazolidine, piperazine can be 1-, 2-, 3- or 4-piperazine, tetrahydrofuran can be 1- or 2-tetrahydrofuran, oxazolidine can be 2-, 3-, 4- or 5-oxazolidine, isoxazolidine can be 2-, 3-, 4- or 5-isoxazolidine, thiazolidine can be 2-, 3-, 4- or 5-thiazolidine, isothiazolidine can be 2-, 3-, 4- or 5-isothiazolidine, and morpholine can be 2-, 3- or 4-morpholine.

As used herein, the terms “halo” and “halogen,” by themselves or as part of another substituent, refer to a fluorine, chlorine, bromine, or iodine atom.

As used herein, the term “carbonyl,” by itself or as part of another substituent, refers to —C(O)—, i.e., a carbon atom double-bonded to oxygen and bound to two other groups in the moiety having the carbonyl.

As used herein, the term “amino” refers to a moiety —NR₃, wherein each R₃ group is H or alkyl. An amino moiety can be ionized to form the corresponding ammonium cation.

As used herein, the term “hydroxy” refers to the moiety —OH.

As used herein, the term “cyano” refers to a carbon atom triple-bonded to a nitrogen atom, i.e., the moiety —CN.

As used herein, the term “carboxy” refers to the moiety —C(O)OH. A carboxy moiety can be ionized to form the corresponding carboxylate anion.

As used herein, the term “amido” refers to a moiety —NR₃C(O)— or —C(O)N(R₃)₂, wherein each R₃ group is H or alkyl.

As used herein, the term “nitro” refers to the moiety —NO₂.

As used herein, the term “oxo” refers to an oxygen atom that is double-bonded to a compound, i.e., O═.

As used herein, each of the symbol “ [symbol] ” and the dashed line (“ [symbol] ”) defines the location at which the designated structure is bound (for example, each of the symbol “ [symbol] ” and the dashed line (“ [symbol] ”) designates the location of the bond made between an adjuvant and a linker.

As used herein, when the term “optionally present” is used to refer to a chemical structure, and when that chemical structure is not present, the bond originally made to the chemical structure is made directly to the adjacent atom.

As used herein, the term “linker” refers to a functional group that covalently bonds two or more moieties in a compound or material. For example, the linking moiety can serve to covalently bond an adjuvant moiety to an antibody in an immunoconjugate.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement; remission; diminishing of symptoms or making the symptom, injury, pathology, or condition more tolerable to the patient; reduction in the rate of symptom progression; decreasing the frequency or duration of the symptom or condition; or, in some situations, preventing the onset of the symptom. The treatment or amelioration of symptoms can be based on any objective or subjective parameter, including, for example, the result of a physical examination.

The terms “cancer,” “neoplasm,” and “tumor” are used herein to refer to cells which exhibit autonomous, unregulated growth, such that the cells exhibit an aberrant growth phenotype characterized by a significant loss of control over cell proliferation. Cells of interest for detection, analysis, and/or treatment in the context of the invention include cancer cells (e.g., cancer cells from an individual with cancer), malignant cancer cells, pre-metastatic cancer cells, metastatic cancer cells, and non-metastatic cancer cells. Cancers of virtually every tissue are known. The phrase “cancer burden” refers to the quantum of cancer cells or cancer volume in a subject. Reducing cancer burden accordingly refers to reducing the number of cancer cells or the cancer cell volume in a subject. The term “cancer cell” as used herein refers to any cell that is a cancer cell (e.g., from any of the cancers for which an individual can be treated, e.g., isolated from an individual having cancer) or is derived from a cancer cell, e.g., clone of a cancer cell. For example, a cancer cell can be from an established cancer cell line, can be a primary cell isolated from an individual with cancer, can be a progeny cell from a primary cell isolated from an individual with cancer, and the like. In some embodiments, the term can also refer to a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, and myelomas, and circulating cancers such as leukemias.

As used herein, the term “cancer” includes any form of cancer, including but not limited to, solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, and neuroendocrine) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions.

Carcinomas are malignancies that originate in the epithelial tissues. Epithelial cells cover the external surface of the body, line the internal cavities, and form the lining of glandular tissues. Examples of carcinomas include, but are not limited to, adenocarcinoma (cancer that begins in glandular (secretory) cells such as cancers of the breast, pancreas, lung, prostate, and colon) adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, and skin.

Soft tissue tumors are a highly diverse group of rare tumors that are derived from connective tissue. Examples of soft tissue tumors include, but are not limited to, alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typical lipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor; mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DF SP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; pleomorphic adenoma of soft tissue; and neoplasias derived from fibroblasts, myofibroblasts, histiocytes, vascular cells/endothelial cells, and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to, askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; and undifferentiated pleomorphic sarcoma).

A teratoma is a type of germ cell tumor that may contain several different types of tissue (e.g., can include tissues derived from any and/or all of the three germ layers: endoderm, mesoderm, and ectoderm), including, for example, hair, muscle, and bone. Teratomas occur most often in the ovaries in women, the testicles in men, and the tailbone in children.

Melanoma is a form of cancer that begins in melanocytes (cells that make the pigment melanin). Melanoma may begin in a mole (skin melanoma), but can also begin in other pigmented tissues, such as in the eye or in the intestines.

Leukemias are cancers that start in blood-forming tissue, such as the bone marrow, and cause large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to, Acute myeloid leukemia (AML), Acute lymphoblastic leukemia (ALL), Chronic myeloid leukemia (CIVIL), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One category of lymphoma is Hodgkin lymphoma (HL), which is marked by the presence of a type of cell called the Reed-Sternberg cell. There are currently 6 recognized types of HL. Examples of Hodgkin lymphomas include nodular sclerosis classical Hodgkin lymphoma (CHL), mixed cellularity CHL, lymphocyte-depletion CHL, lymphocyte-rich CHL, and nodular lymphocyte predominant HL.

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL. Examples of non-Hodgkin lymphomas include, but are not limited to, AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to, gliomas (e.g., glioblastomas, astrocytomas, oligodendrogliomas, ependymomas, and the like), meningiomas, pituitary adenomas, and vestibular schwannomas, primitive neuroectodermal tumors (medulloblastomas).

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, and invasion of surrounding or distant tissues or organs, such as lymph nodes.

As used herein, the phrases “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs, therefore, tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part that is not directly connected to the organ of the original cancerous tumor. Metastasis can also be defined as several steps of a process, such as the departure of cancer cells from an original tumor site, and migration and/or invasion of cancer cells to other parts of the body.

As used herein the phrases “effective amount” and “therapeutically effective amount” refer to a dose of a substance such as an immunoconjugate that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); Goodman & Gilman's The Pharmacological Basis of Therapeutics, 11^{th} Edition (McGraw-Hill, 2006); and Remington: The Science and Practice of Pharmacy, 22^{nd} Edition, (Pharmaceutical Press, London, 2012)).

As used herein, the terms “recipient,” “individual,” “subject,” “host,” and “patient,” are used interchangeably and refer to any mammalian subject for whom diagnosis, treatment, or therapy is desired (e.g., humans). “Mammal” for purposes of treatment refers to any animal classified as a mammal, including humans, domestic and farm animals, and zoo, sports, or pet animals, such as dogs, horses, cats, cows, sheep, goats, pigs, camels, etc. In certain embodiments, the mammal is human.

The phrase “synergistic adjuvant” or “synergistic combination” in the context of this invention includes the combination of two immune modulators such as a receptor agonist, cytokine, and adjuvant polypeptide, that in combination elicit a synergistic effect on immunity relative to either administered alone. Particularly, the immunoconjugates disclosed herein comprise synergistic combinations of an adjuvant that is a TLR agonist and an antibody. These synergistic combinations upon administration elicit a greater effect on immunity, e.g., relative to when the antibody or adjuvant is administered in the absence of the other moiety. Further, a decreased amount of the immunoconjugate may be administered (as measured by the total number of antibodies or the total number of adjuvants administered as part of the immunoconjugate) compared to when either the antibody or adjuvant is administered alone.

As used herein, the term “administering” refers to parenteral, intravenous, intraperitoneal, intramuscular, intratumoral, intralesional, intranasal, or subcutaneous administration, oral administration, administration as a suppository, topical contact, intrathecal administration, or the implantation of a slow-release device, e.g., a mini-osmotic pump, to the subject.

The terms “about” and “around,” as used herein to modify a numerical value, indicate a close range surrounding the numerical value. Thus, if “X” is the value, “about X” or “around X” indicates a value of from 0.9× to 1.1×, e.g., from 0.95× to 1.05× or from 0.99× to 1.01×. A reference to “about X” or “around X” specifically indicates at least the values X, 0.95×, 0.96×, 0.97×, 0.98×, 0.99×, 1.01×, 1.02×, 1.03×, 1.04×, and 1.05×. Accordingly, “about X” and “around X” are intended to teach and provide written description support for a claim limitation of, e.g., “0.98×.”

Antibody Adjuvant Conjugates

In some embodiments, the immunoconjugate is of formula:

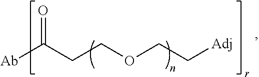

or pharmaceutically acceptable salt thereof, wherein subscript r is an integer from 1 to 10, subscript n is an integer from about 2 to about 50 (e.g., about 2 to about 25, about 2 to about 16, about 6 to about 50, about 6 to about 25, about 6 to about 16, about 8 to about 50, about 8 to about 25, about 8 to about 16, or about 8 to about 12), “Adj” is an adjuvant moiety, and “Ab” is an antibody construct that has an antigen binding domain that binds epidermal growth factor receptor (“EGFR”). In certain embodiments, “Ab” is cetuximab (also known as ERBITUX™), a biosimilar thereof, or a biobetter thereof. In other embodiments, “Ab” is panitumumab (also known as VECTIBIX™), a biosimilar thereof, or a biobetter thereof. In certain embodiments, “Ab” is necitumumab (also known as PORTRAZZA™), a biosimilar thereof, or a biobetter thereof.

In certain embodiments, the immunoconjugate is of formula:

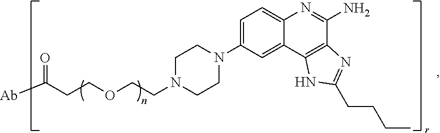

or pharmaceutically acceptable salt thereof, wherein subscript r is an integer from 1 to 10, subscript n is an integer from about 2 to about 50 (e.g., about 2 to about 25, about 2 to about 16, about 6 to about 50, about 6 to about 25, about 6 to about 16, about 8 to about 50, about 8 to about 25, about 8 to about 16, or about 8 to about 12), and “Ab” is an antibody construct that has an antigen binding domain that binds epidermal growth factor receptor (“EGFR”). In certain embodiments, “Ab” is cetuximab (also known as ERBITUX™) a biosimilar thereof, or a biobetter thereof. In other embodiments, “Ab” is panitumumab (also known as VECTIBIX™), a biosimilar thereof, or a biobetter thereof. In certain embodiments, “Ab” is necitumumab (also known as PORTRAZZA™), a biosimilar thereof, or a biobetter thereof.

Generally, the immunoconjugates of the invention comprise about 1 to about 10 adjuvants linked via a polyethylene glycol (“PEG”) linker, as designated with subscript “r”. Each of the adjuvants linked via a PEG linker are conjugated to the antibody construct at an amine of a lysine residue of the antibody construct. In some embodiments, r is an integer from about 2 to about 10 (e.g., about 2 to about 9, about 3 to about 9, about 4 to about 9, about 5 to about 9, about 6 to about 9, about 3 to about 8, about 3 to about 7, about 3 to about 6, about 4 to about 8, about 4 to about 7, about 4 to about 6, about 5 to about 6, about 1 to about 6, about 1 to about 4, about 2 to about 4, or about 1 to about 3). Accordingly, the immunoconjugates can have (i.e., subscript “r” can be) 1, 2, 3, 4, 5, 6, 7, 8, 9, or 10 adjuvants linked via a PEG linker. In preferred embodiments, the immunoconjugates have (i.e., subscript “r” can be) 1, 2, 3, or 4 adjuvants linked via a PEG linker. The desirable adjuvant set to antibody construct ratio can be determined by a skilled artisan depending on the desired effect of the treatment.

Generally, the immunoconjugates of the invention comprise about 2 to about 50 (e.g., about 2 to about 25, about 2 to about 16, about 6 to about 50, about 6 to about 25, about 6 to about 16, about 8 to about 50, about 8 to about 25, about 8 to about 16, or about 8 to about 12) ethylene glycol units, as designated with subscript “n”. Accordingly, the immunoconjugates of the invention can comprise at least 2 ethylene glycol groups (e.g., at least 3 ethylene glycol groups, at least 4 ethylene glycol groups, at least 5 ethylene glycol groups, at least 6 ethylene glycol groups, at least 7 ethylene glycol groups, at least 8 ethylene glycol groups, at least 9 ethylene glycol groups, at least 10 ethylene glycol groups, at least 11 ethylene glycol groups, at least 12 ethylene glycol groups, at least 13 ethylene glycol groups, at least 14 ethylene glycol groups, at least 15 ethylene glycol groups, at least 16 ethylene glycol groups, at least 17 ethylene glycol groups, at least 18 ethylene glycol groups, at least 19 ethylene glycol groups, at least 20 ethylene glycol groups, at least 21 ethylene glycol groups, at least 22 ethylene glycol groups, at least 23 ethylene glycol groups, at least 24 ethylene glycol groups, or at least 25 ethylene glycol groups. Accordingly, the immunoconjugate can comprise from about 2 to about 25 ethylene glycol units, for example, from about 6 to about 25 ethylene glycol units, from about 6 to about 16 ethylene glycol units, from about 8 to about 25 ethylene glycol units, from about 8 to about 16 ethylene glycol units, or from about 8 to about 12 ethylene glycol units. In certain embodiments, the immunoconjugate comprises a di(ethylene glycol) group, a tri(ethylene glycol) group, a tetra(ethylene glycol) group, 5 ethylene glycol groups, 6 ethylene glycol groups, 7 ethylene glycol groups, 8 ethylene glycol groups, 9 ethylene glycol groups, 10 ethylene glycol groups, 11 ethylene glycol groups, 12 ethylene glycol groups, 13 ethylene glycol groups, 14 ethylene glycol groups, 15 ethylene glycol groups, 16 ethylene glycol groups, 24 ethylene glycol groups, or 25 ethylene glycol groups.

The PEG linker is linked to the antibody construct that has an antigen binding domain that binds EGFR (e.g., cetuximab or a biosimilar of cetuximab) via an amine of a lysine residue of the antibody construct. Accordingly, the immunoconjugates of the invention can be represented by the following formula:

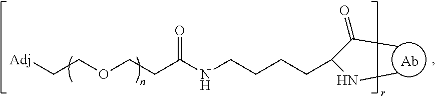

wherein “Adj” is an adjuvant moiety, subscript n is an integer from about 2 to about 50 (e.g., about 2 to about 25, about 2 to about 16, about 6 to about 50, about 6 to about 25, about 6 to about 16, about 8 to about 50, about 8 to about 25, about 8 to about 16, or about 8 to about 12), and

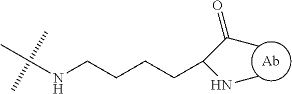

is an antibody construct that has an antigen binding domain that binds EGFR with residue

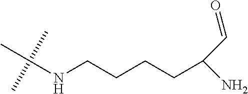

representing a lysine residue of the antibody construct, wherein “ [symbol] ” represents a point of attachment to the linker.

Immunoconjugates as described herein can provide an unexpectedly increased activation response of an APC. This increased activation can be detected in vitro or in vivo. In some embodiments, the increased APC activation can be detected in the form of a reduced time to achieve a specified level of APC activation. For example, in an in vitro assay, % APC activation can be achieved at an equivalent dose with an immunoconjugate within 1%, 10%, 20%, 30%, 40%, or 50% of the time required to obtain the same or similar percentage of APC activation with a mixture of unconjugated antibody construct and the adjuvant, under otherwise identical concentrations and conditions. In some embodiments, an immunoconjugate can activate APCs (e.g., dendritic cells) and/or NK cells in a reduced amount of time. For example, in some embodiments, a mixture of unconjugated antibody construct and the adjuvant can activate APCs (e.g., dendritic cells) and/or NK cells and/or induce dendritic cell differentiation after incubation with the mixture for 2, 3, 4, 5, 1-5, 2-5, 3-5, or 4-7 days, while, in contrast immunoconjugates described herein can activate and/or induce differentiation within 4 hours, 8 hours, 12 hours, 16 hours, or 1 day, under otherwise identical concentrations and conditions. Alternatively, the increased APC activation can be detected in the form of a reduced concentration of immunoconjugate required to achieve an amount (e.g., percent APCs), level (e.g., as measured by a level of upregulation of a suitable marker) or rate (e.g., as detected by a time of incubation required to activate) of APC activation.

In some embodiments, the immunoconjugates of the invention provide more than a 5% increase in activity compared to a mixture of unconjugated antibody construct and the adjuvant, under otherwise identical conditions. In other embodiments, the immunoconjugates of the invention provide more than a 10%, 15%, 20%, 25%, 30%, 35%, 40%, 45%, 50%, 55%, 60%, 65%, or 70% increase in activity compared to a mixture of unconjugated antibody construct and the adjuvant, under otherwise identical conditions. The increase in activity can be assessed by any suitable means, many of which are known to those ordinarily skilled in the art and can include myeloid activation, assessment by cytokine secretion, or a combination thereof.

In a related aspect, the invention provides a composition comprising a plurality of immunoconjugates as described above. Accordingly, immunoconjugates of the invention can have an average adjuvant to antibody construct ratio of about 0.4, 0.6, 0.8, 1, 1.2, 1.4, 1.6, 1.8, 2, 2.2, 2.4, 2.6, 2.8, 3, 3.2, 3.4, 3.6, 3.8, 4.0, 4.2, 4.4, 4.6, 4.8, 5.0, 5.2, 5.4, 5.6, 5.8, 6.0, 6.2, 6.4, 6.6, 6.8, 7, 7.2, 7.4, 7.6, 7.8, 8, 8.2, 8.4, 8.6, 8.8, 9, 9.2, 9.4, 9.6, 9.8, or 10. A skilled artisan will recognize that the number of adjuvant conjugated to the antibody construct may vary from immunoconjugate to immunoconjugate in a composition comprising multiple immunoconjugates of the invention, and, thus, the adjuvant to antibody construct (e.g., antibody) ratio can be measured as an average. The adjuvant set to antibody construct (e.g., antibody) ratio can be assessed by any suitable means, many of which are known in the art.

In some embodiments, the invention provides an immunoconjugate of formula:

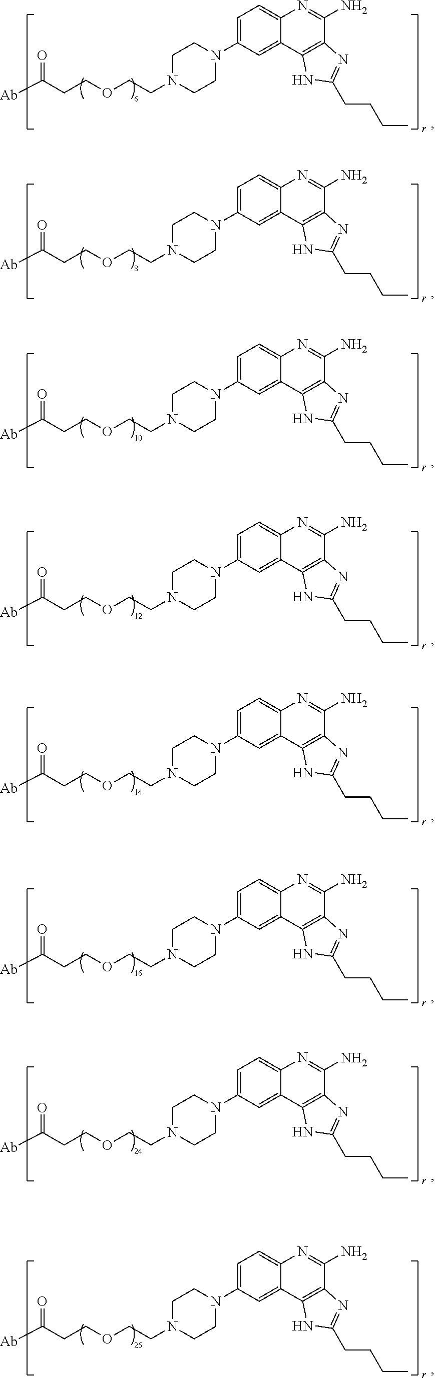

or pharmaceutically acceptable salt thereof, wherein subscript r is an integer from 1 to 10 and “Ab” is an antibody construct that has an antigen binding domain that binds EGFR.

In certain embodiments, the invention provides an immunoconjugate of formula:

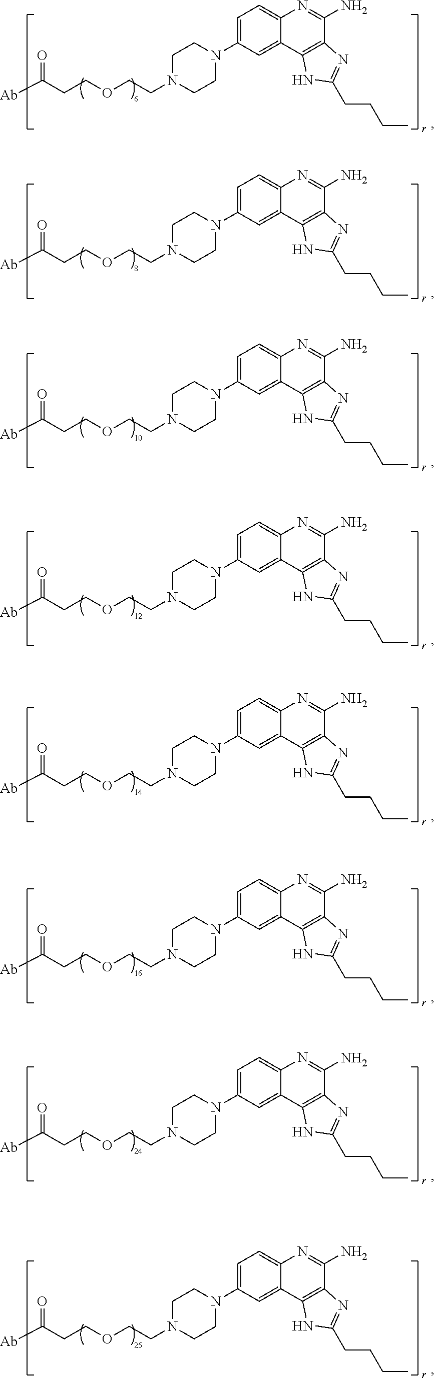

or pharmaceutically acceptable salt thereof, wherein subscript r is an integer from 1 to 10 and “Ab” is cetuximab (also known as ERBITUX™).

In certain embodiments, the invention provides an immunoconjugate of formula:

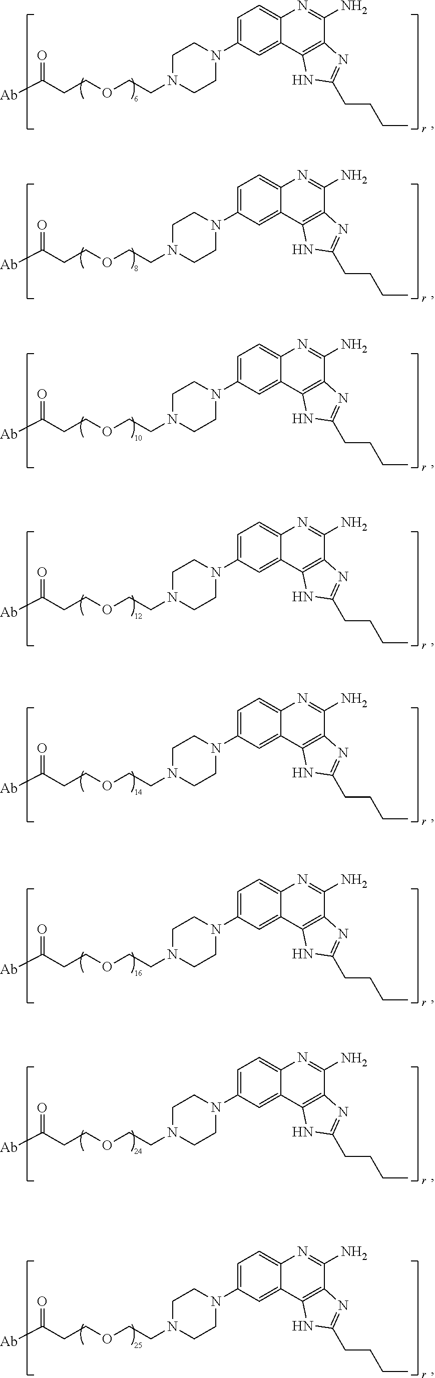

or pharmaceutically acceptable salt thereof, wherein subscript r is an integer from 1 to 10 and “Ab” is panitumumab (also known as VECTIBIX™).

In certain embodiments, the invention provides an immunoconjugate of formula:

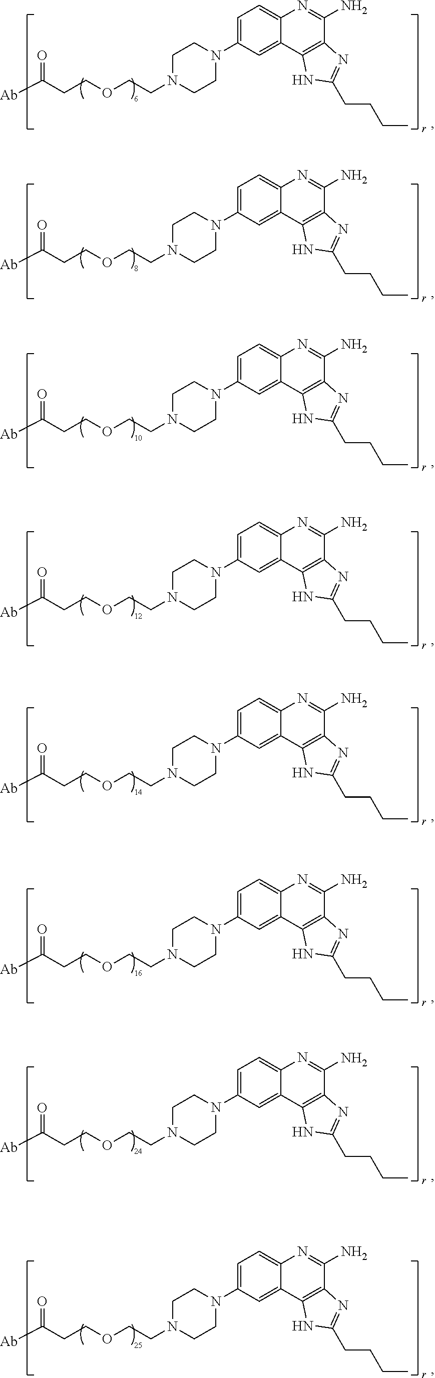

or pharmaceutically acceptable salt thereof, wherein subscript r is an integer from 1 to 10 and “Ab” is necitumumab (also known as PORTRAZZA™).

In other embodiments, the invention provides an immunoconjugate of formula:

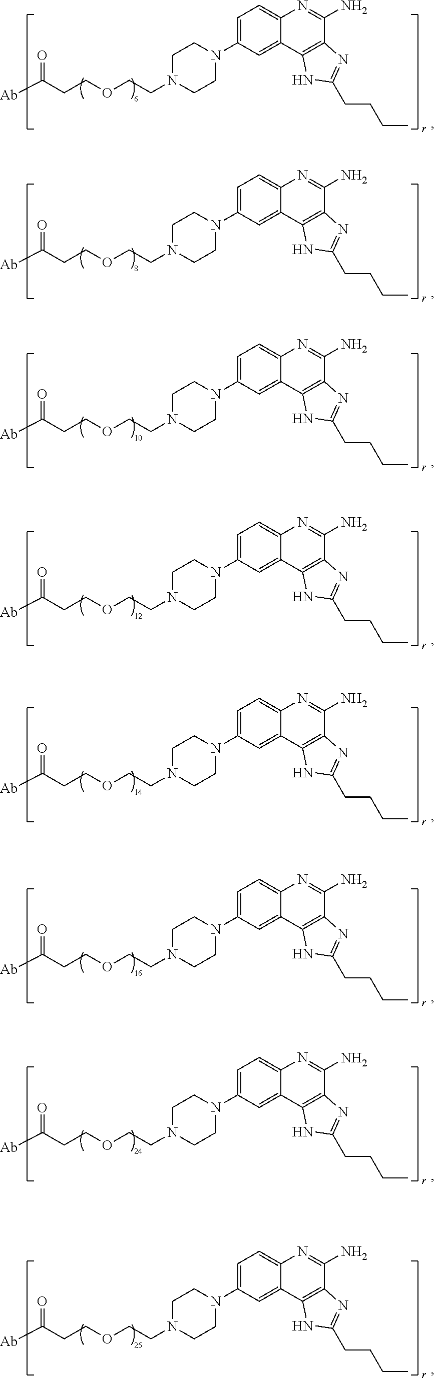

or pharmaceutically acceptable salt thereof, wherein subscript r is an integer from 1 to 10 and “Ab” is a biosimilar or biobetter of (1) cetuximab, (2) panitumumab, or (3) necitumumab, a biosimilar thereof, or a biobetter thereof. For example, “Ab” can be STI-001, RPH-002, CMAB009, ONS-1055, MabionEGFR, HLX-05, HLX05, CT-P15, KN-005, ABP-494, AP-087, EMD72000 (also known as matuzumab), tomuzotuximab (also known as CETUGEX™), GC1118, SYN004, SCT200, or HLX-07.

### Adjuvants

The adjuvant moiety described herein is a compound that elicits an immune response (i.e., an immunostimulatory agent). In some embodiments, the adjuvant moiety is a pattern recognition receptor (“PRR”) agonist. As used herein, the terms “pattern recognition receptor” and “PRR” refer to any member of a class of conserved mammalian proteins which recognizes pathogen-associated molecular patterns (“PAMPs”) or damage-associated molecular patterns (“DAMPs”), and acts as a key signaling element in innate immunity. PRRs are divided into membrane-bound PRRs, cytoplasmic PRRs, and secreted PRRs. Examples of membrane-bound PRRs include Toll-like receptors (“TLRs”) and C-type lectin receptors (“CLRs”). Examples of cytoplasmic PRRs include NOD-like receptors (“NLRs”) and Rig-I-like receptors (“RLRs”).

Generally, the adjuvant moiety described herein is a TLR agonist. Suitable TLR agonists include TLR1, TLR2, TLR3, TLR4, TLR5, TLR6, TLR7, TLR8, TLR9, TLR10, TLR11, or any combination thereof (e.g., TLR7/8 agonists). TLRs are type-I transmembrane proteins that are responsible for the initiation of innate immune responses in vertebrates. TLRs recognize a variety of pathogen-associated molecular patterns from bacteria, viruses, and fungi and act as a first line of defense against invading pathogens. TLRs elicit overlapping yet distinct biological responses due to differences in cellular expression and in the signaling pathways that they initiate. Once engaged (e.g., by a natural stimulus or a synthetic TLR agonist), TLRs initiate a signal transduction cascade leading to activation of nuclear factor-κB (NF-κB) via the adapter protein myeloid differentiation primary response gene 88 (MyD88) and recruitment of the IL-1 receptor associated kinase (IRAK). Phosphorylation of IRAK then leads to recruitment of TNF-receptor associated factor 6 (TRAF6), which results in the phosphorylation of the NF-κB inhibitor I-κB. As a result, NF-κB enters the cell nucleus and initiates transcription of genes whose promoters contain NF-κB binding sites, such as cytokines. Additional modes of regulation for TLR signaling include TIR-domain containing adapter-inducing interferon-β (TRIF)-dependent induction of TNF-receptor associated factor 6 (TRAF6) and activation of MyD88 independent pathways via TRIF and TRAF3, leading to the phosphorylation of interferon response factor three (IRF3). Similarly, the MyD88 dependent pathway also activates several IRF family members, including IRF5 and IRF7 whereas the TRIF dependent pathway also activates the NF-κB pathway.

Typically, the adjuvant moiety described herein is a TLR7 and/or TLR8 agonist. TLR7 and TLR8 are both expressed in monocytes and dendritic cells. In humans, TLR7 is also expressed in plasmacytoid dendritic cells (pDCs) and B cells. TLR8 is expressed mostly in cells of myeloid origin, i.e., monocytes, granulocytes, and myeloid dendritic cells. TLR7 and TLR8 are capable of detecting the presence of “foreign” single-stranded RNA within a cell, as a means to respond to viral invasion. Treatment of TLR8-expressing cells, with TLR8 agonists can result in production of high levels of IL-12, IFN-γ, IL-1, TNF-α, IL-6, and other inflammatory cytokines. Similarly, stimulation of TLR7-expressing cells, such as pDCs, with TLR7 agonists can result in production of high levels of IFN-α and other inflammatory cytokines. TLR7/TLR8 engagement and resulting cytokine production can activate dendritic cells and other antigen-presenting cells, driving diverse innate and acquired immune response mechanisms leading to tumor destruction.

In certain embodiments, at least one adjuvant moiety is of formula:

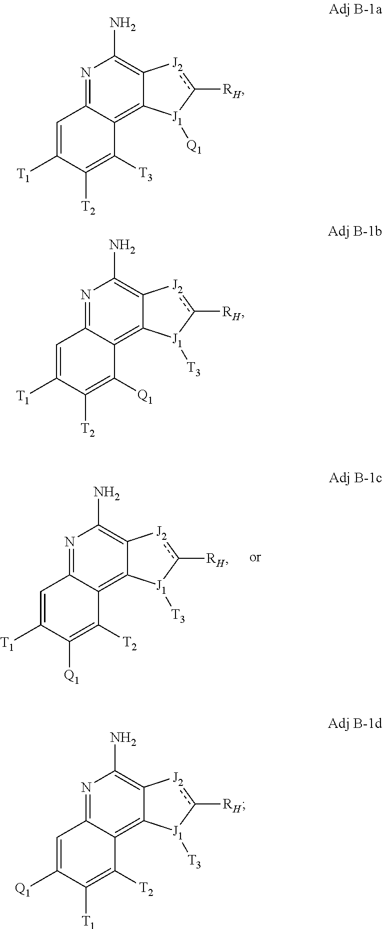

wherein

J₁ is CH or N,

J₂ is CH, CH₂, N, NH, O, or S,

Q₁ is of the formula:

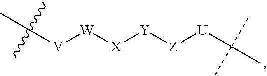

T₁, T₂, T₃, and R_{H} independently are of the formula:

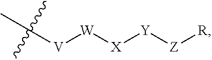

each V is optionally present and independently is —O—, —S—, —NH—, —NR—, or —CO—, each W is optionally present and independently is a linear or branched, saturated or unsaturated, divalent C₁-C₈ alkyl,

each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

each Y is optionally present and independently is —CO— or a linear or branched, saturated or unsaturated, divalent C₁-C₈ alkyl, each Z is optionally present and independently is —O—, —S—, —NH—, or —NR—,

U is optionally present and is

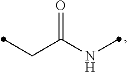

each R independently is hydrogen, halogen (e.g., fluorine, chlorine, bromine, or iodine), nitrile, —COOH, or linear or branched, saturated or unsaturated C₁-C₄ alkyl,

“ [symbol] ” represents a single bond or a double bond,

the wavy line (“ [symbol] ”) represents a point of attachment of Q₁, T₁, T₂, T₃, and R_{H}, the dot (“●”) represents a point of attachment of U, and the dashed line (“ [symbol] ”) represents a point of attachment of the adjuvant moiety.

In certain embodiments, at least one adjuvant moiety is of formula:

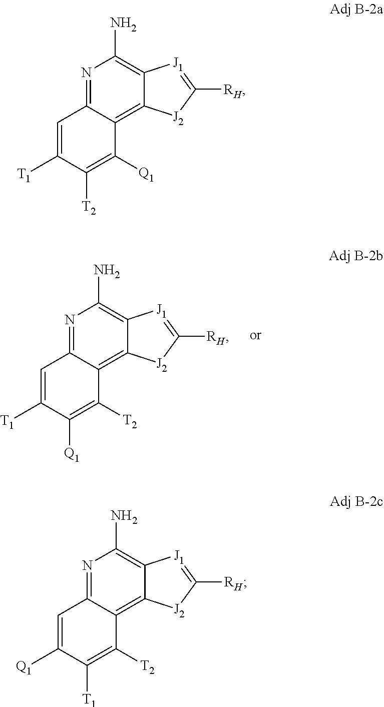

wherein

J₁ is CH or N,

J₂ is CH₂, NH, O, or S,

Q₁ is of the formula:

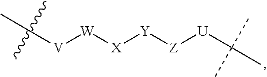

T₁, T₂, and R_{H} independently are of the formula:

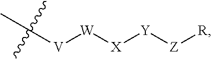

each V is optionally present and independently is —O—, —S—, —NH—, —NR—, or —CO—,

each W is optionally present and independently is a linear or branched, saturated or unsaturated, divalent C₁-C₈ alkyl,

each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

each Y is optionally present and independently is —CO— or a linear or branched, saturated or unsaturated, divalent C₁-C₈ alkyl,

each Z is optionally present and independently is —O—, —S—, —NH— or —NR—,

U is optionally present and is

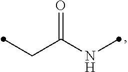

each R independently is hydrogen, halogen (e.g., fluorine, chlorine, bromine, or iodine), nitrile, —COOH, or a linear or branched, saturated or unsaturated C₁-C₄ alkyl, the wavy line (“ [symbol] ”) represents a point of attachment of Q₁, T₁, T₂, and R_{H},

[symbol]

the dot (“●”) represents a point of attachment of U, and

the dashed line (“ [symbol] ”) represents a point of attachment of the adjuvant moiety.

In certain embodiments, at least one adjuvant moiety is of formula:

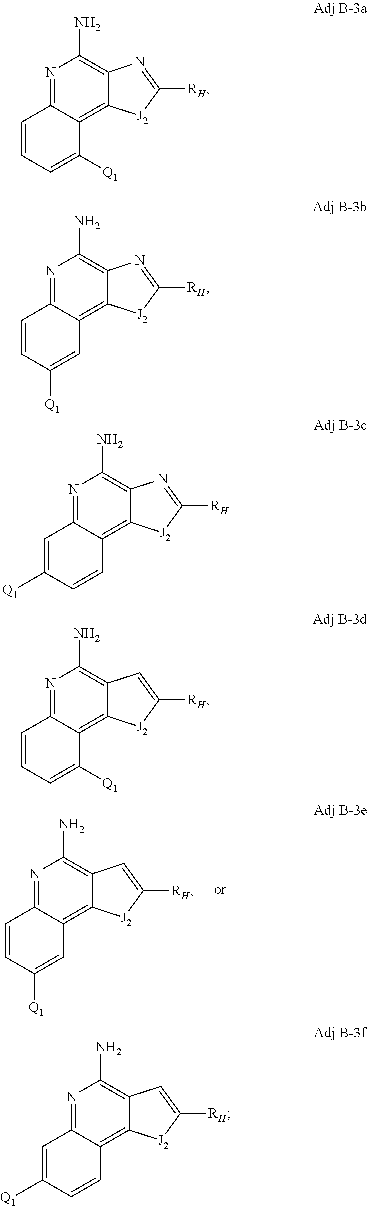

wherein

J₂ is CH₂, NH, O, or S,

Q₁ is of the formula:

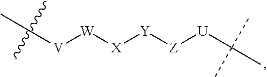

R_{H} is of the formula:

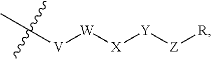

each V is optionally present and independently is —O—, —S—, —NH—, —NR—, or —CO—,

each W is optionally present and independently is a linear or branched, saturated or unsaturated, divalent C₁-C₈ alkyl,

each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

each Y is optionally present and independently is —CO— or a linear or branched, saturated or unsaturated, divalent C₁-C₈ alkyl,

each Z is optionally present and independently is —O—, —S—, —NH—, or —NR—,

U is optionally present and is

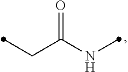

each R independently is hydrogen, halogen (e.g., fluorine, chlorine, bromine, or iodine), nitrile, —COOH, or a linear or branched, saturated or unsaturated C₁-C₄ alkyl,

the wavy line (“ [symbol] ”) represents a point of attachment of Q₁ and R_{H},

the dot (“●”) represents a point of attachment of U, and

the dashed line (“ [symbol] ”) represents a point of attachment of the adjuvant moiety.

In certain embodiments at least one adjuvant moiety is of formula:

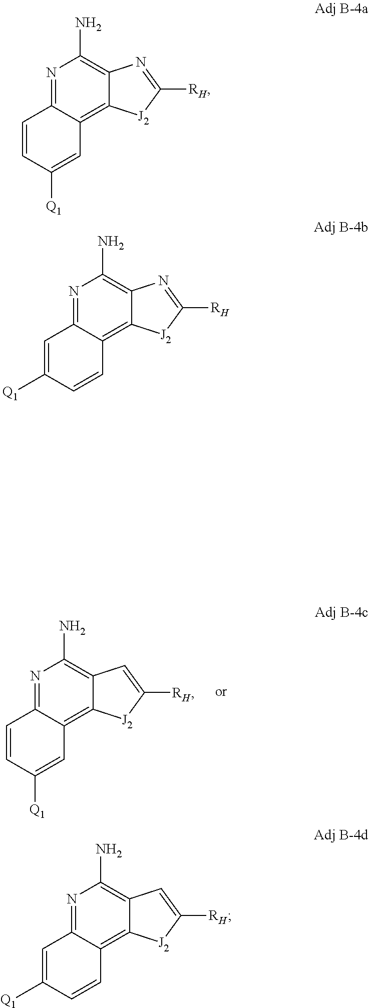

wherein

J₂ is CH₂, NH, O, or S,

Q₁ is of the lbrmula:

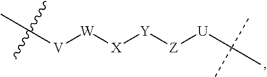

R_{H} is of the formula:

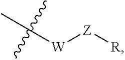

V is optionally present and is —O—, —S—, —NH—, —NR—, or —CO—,

each W is optionally present and independently is a linear or branched, saturated or unsaturated, divalent C₁-C₈ alkyl,

X is optionally present and is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—, Y is optionally present and is —CO— or a linear or branched, saturated or unsaturated, divalent C₁-C₈ alkyl,

each Z is optionally present and independently is —O—, —S—, —NH—, or —NR—,

U is optionally present and is

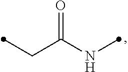

each R independently is hydrogen, halogen (e.g., fluorine, chlorine, bromine, or iodine), nitrile, —COOH, or a linear or branched, saturated or unsaturated C₁-C₄ alkyl,

the wavy line (“ [symbol] ”) represents a point of attachment of Q₁ and R_{H},

the dot (“●”) represents a point of attachment of U, and

the dashed line (“ [symbol] ”) represents a point of attachment of the adjuvant moiety.

In preferred embodiments, at least one adjuvant moiety is of formula:

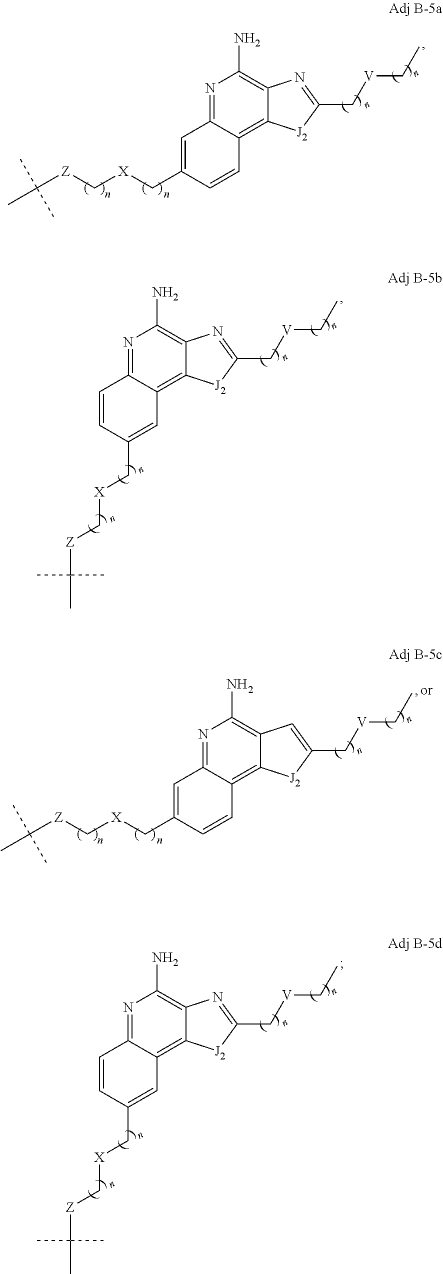

wherein

J₂ is CH₂, NH, O, or S,

V is optionally present and is —O—, —S—, —NH—, —NR—, or —CO—,

X is optionally present and is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

Z is optionally present and is —O—, —S—, —NH— or —NR—,

provided that at least X or Z is present,

each R independently is hydrogen, halogen (e.g., fluorine, chlorine, bromine, or iodine), nitrile, —COOH, or a linear or branched, saturated or unsaturated C₁-C₄ alkyl,

each n independently is an integer from 0 to 4, and

the dashed line (“ [symbol] ”) represents a point of attachment of the adjuvant moiety.

More preferably, at least one adjuvant moiety is of formula:

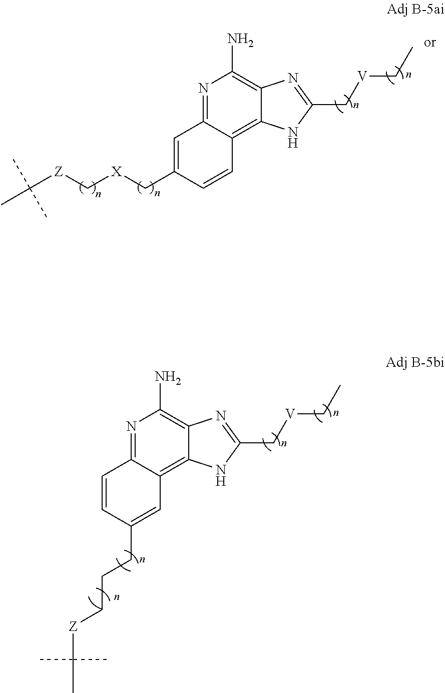

wherein

V is optionally present and is —O—, —S—, —NH—, —NR—, or —CO—,

X is optionally present and is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

Z is optionally present and is —O—, —S—, —NH—, or —NR—,

provided that at least X or Z is present,

each R independently is hydrogen, halogen (e.g., fluorine, chlorine, bromine, or iodine), nitrile, —COOH, or a linear or branched, saturated or unsaturated C₁-C₄ alkyl,

each n independently is an integer from 0 to 4, and

the dashed line (“ [symbol] ”) represents a point of attachment.

In some embodiments, at least one adjuvant moiety is of formula:

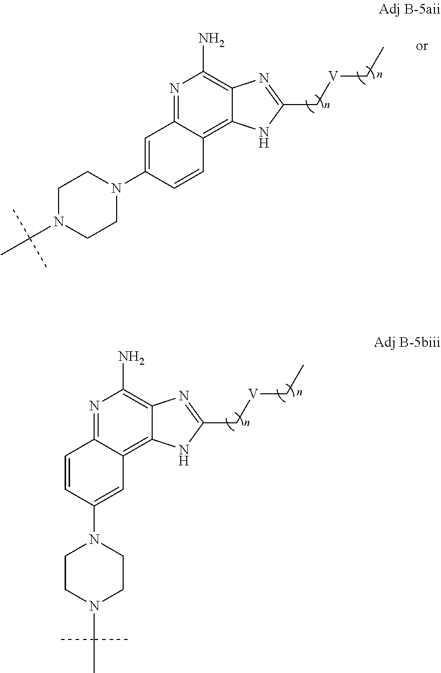

wherein

V is optionally present and is —O—, —S—, —NH—, —NR—, or —CO—,

R is hydrogen, halogen (e.g., fluorine, chlorine, bromine, or iodine), nitrile, —COOH, or a linear or branched, saturated or unsaturated C₁-C₄ alkyl,

each n independently is an integer from 0 to 4, and

the dashed line (“ [symbol] ”) represents a point of attachment of the adjuvant moiety.

In preferred embodiments, the immunoconjugates of the invention comprise an adjuvant moiety of formula:

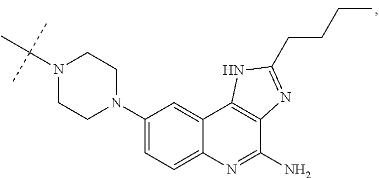

wherein the dashed line (“ [symbol] ”) represents a point of attachment of the adjuvant moiety to the linker.

Antigen Binding Domain and Fc Domain

The immunoconjugates of the invention comprise an antibody construct that comprises an antigen binding domain that binds EGFR. In some embodiments, there antibody construct further comprises an Fc domain. In some embodiments, the antibody construct further comprises a targeting binding domain. In certain embodiments, the antibody construct is an antibody. In certain embodiments, the antibody construct is a fusion protein.

The antigen binding domain can be a single-chain variable region fragment (scFv). A single-chain variable region fragment (scFv), which is a truncated Fab fragment including the variable (V) domain of an antibody heavy chain linked to a V domain of a light antibody chain via a synthetic peptide, can be generated using routine recombinant DNA technology techniques. Similarly, disulfide-stabilized variable region fragments (dsFv) can be prepared by recombinant DNA technology.

The antibodies in the immunoconjugates can be allogeneic antibodies. The terms “allogeneic antibody” or “alloantibody” refer to an antibody that is not from the individual in question (e.g., an individual with a tumor and seeking treatment), but is from the same species, or is from a different species, but has been engineered to reduce, mitigate, or avoid recognition as a xeno-antibody (e.g., non-self). For example, the “allogeneic antibody” can be a humanized antibody. One skilled in the art is knowledgeable regarding how to engineer a non-human antibody to avoid recognition as a xeno-antibody. Unless specifically stated otherwise, “antibody” and “allogeneic antibodies,” as used herein, refer to immunoglobulin G (IgG).

If a cancer cell of a human individual is contacted with an antibody that was not generated by that same person (e.g., the antibody was generated by a second human individual, the antibody was generated by another species such as a mouse, the antibody is a humanized antibody that was generated by another species, etc.), then the antibody is considered to be allogeneic (relative to the first individual). A humanized mouse monoclonal antibody that recognizes a human antigen (e.g., a cancer-specific antigen, an antigen that is enriched in and/or on cancer cells, etc.) is considered to be an “alloantibody” (an allogeneic antibody). In some embodiments, the antibody is a polyclonal allogeneic IgG antibody.

In some embodiments where the antibodies in the immunoconjugates comprise IgGs from serum, the target antigens for some (e.g., greater than 0% but less than 50%), half, most (greater than 50% but less than 100%), or even all of the antibodies (i.e., IgGs from the serum) will be unknown. However, the chances are high that at least one antibody in the mixture will recognize the target antigen of interest because such a mixture contains a wide variety of antibodies specific for a wide variety of target antigens.

In some embodiments where the antibodies in the immunoconjugates comprise IgAs from serum, the target antigens for some (e.g., greater than 0% but less than 50%), half, most (greater than 50% but less than 100%), or even all of the antibodies (i.e., IgAs from the serum) will be unknown. However, the chances are high that at least one antibody in the mixture will recognize the target antigen of interest because such a mixture contains a wide variety of antibodies specific for a wide variety of target antigens.

In some embodiments, the antibody in the immunoconjugates includes intravenous immunoglobulin (IVIG) and/or antibodies from (e.g., enriched from or purified from, such as affinity purified from) IVIG. IVIG is a blood product that contains IgG (immunoglobulin G) pooled from the plasma (e.g., in some embodiments without any other proteins) from many (e.g., sometimes over 1,000 to 60,000) normal and healthy blood donors. IVIG is commercially available. IVIG contains a high percentage of native human monomeric IVIG and has low IgA content. When administered intravenously, IVIG ameliorates several disease conditions. Therefore, the United States Food and Drug Administration (FDA) has approved the use of IVIG for a number of diseases including (1) Kawasaki disease, (2) immune-mediated thrombocytopenia, (3) primary immunodeficiencies, (4) hematopoietic stem cell transplantation (for those older than 20 years), (5) chronic B-cell lymphocytic leukemia, and (6) pediatric HIV type 1 infection. In 2004, the FDA approved the Cedars-Sinai IVIG Protocol for kidney transplant recipients so that such recipients could accept a living donor kidney from any healthy donor, regardless of blood type (ABO incompatible) or tissue match. These and other aspects of IVIG are described, for example, in U.S. Patent Application Publications 2010/0150942, 2004/0101909, 2013/0177574, 2013/0108619, and 2013/0011388; which are hereby incorporated by reference in their entireties.

In some embodiments, the antibody is a monoclonal antibody of a defined subclass (e.g., IgG₁, IgG₂, IgG₃, IgG₄, IgA₁, or IgA₂). If combinations of antibodies are used, the antibodies can be from the same subclass or from different subclasses. Typically, the antibody construct is an IgG₁ antibody. Various combinations of different subclasses, in different relative proportions, can be obtained by those of skill in the art. In some embodiments, a specific subclass or a specific combination of different subclasses can be particularly effective at cancer treatment or tumor size reduction. Accordingly, some embodiments of the invention provide immunoconjugates wherein the antibody is a monoclonal antibody. In some embodiments, the monoclonal antibody is a humanized monoclonal antibody.

In some embodiments, the antibody binds to an antigen of a cancer cell. For example, the antibody can bind to a target antigen that is present at an amount of at least 10, 100, 1,000, 10,000, 100,000, 1,000,000, 2.5×10⁶, 5×10⁶, or 1×10⁷ copies or more on the surface of a cancer cell.

In some embodiments, the antibody binds to an antigen on a cancer or immune cell at a higher affinity than a corresponding antigen on a non-cancer cell. For example, the antibody may preferentially recognize an antigen containing a polymorphism that is found on a cancer or immune cell as compared to recognition of a corresponding wild-type antigen on the non-cancer or non-immune cell. In some embodiments, the antibody binds a cancer or immune cell with greater avidity than a non-cancer or non-immune cell. For example, the cancer or immune cell can express a higher density of an antigen, thereby providing for a higher affinity binding of a multivalent antibody to the cancer or immune cell.

In some embodiments, the antibody does not significantly bind non-cancer antigens (e.g., the antibody binds one or more non-cancer antigens with at least 10, 100, 1.000, 10,000, 100,000, or 1,000,000-fold lower affinity (higher Kd) than the target cancer antigen). In some embodiments, the target cancer antigen to which the antibody binds is enriched on the cancer cell. For example, the target cancer antigen can be present on the surface of the cancer cell at a level that is at least 2, 5, 10, 100, 1,000, 10,000, 100,000, or 1,000,000-fold higher than a corresponding non-cancer cell. In some embodiments, the corresponding non-cancer cell is a cell of the same tissue or origin that is not hyperproliferative or otherwise cancerous. In general, an IgG antibody that specifically binds to an antigen (a target antigen) of a cancer cell preferentially binds to that particular antigen relative to other available antigens. However, the target antigen need not be specific to the cancer cell or even enriched in cancer cells relative to other cells (e.g., the target antigen can be expressed by other cells). Thus, in the phrase “an antibody that specifically binds to an antigen of a cancer cell,” the term “specifically” refers to the specificity of the antibody and not to the uniqueness of the antigen in that particular cell type.

In some embodiments, the antibody is selected from panitumumab, cetuximab, necitumumab, STI-001, RPH-002, CMAB009, ONS-1055, MabionEGFR, HLX-05, HLX05, CT-P15, KN-005, ABP-494, AP-087, EMD72000 (also known as matuzumab), futuximab, modotuximab, tomuzotuximab (also known as CETUGEX™), imgatuzumab, MDX-214, Mab-806, JNJ-6372, ATC-EGFRBi, GC1118, SYN004, SCT200, EMD-55900, ICR-62, HLX-07, or a combination thereof.

Modified Fc Region

In some embodiments, the antibodies in the immunoconjugates contain a modified Fc region, wherein the modification modulates the binding of the Fc region to one or more Fc receptors.

The terms “Fc receptor” or “FcR” refer to a receptor that binds to the Fc region of an antibody. There are three main classes of Fc receptors: (1) FcγR which bind to IgG, (2) FcαR which binds to IgA, and (3) FcεR which binds to IgE. The FcγR family includes several members, such as FcγI (CD64), FcγRIIA (CD32A), FcγRIIB (CD32B), FcγRIIIA (CD16A), and FcγRIIIB (CD16B). The Fey receptors differ in their affinity for IgG and also have different affinities for the IgG subclasses (e.g., IgG1, IgG2, IgG3, and IgG4).

In some embodiments, the antibodies in the immunoconjugates (e.g., antibodies conjugated to at least two adjuvant moieties) contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies in the immunoconjugates contain one or more modifications (e.g., amino acid insertion, deletion, and/or substitution) in the Fc region that reduce the binding of the Fc region of the antibody to FcγRIIB. In some embodiments, the antibodies in the immunoconjugates contain one or more modifications (e.g., amino acid insertion, deletion, and/or substitution) in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies in the immunoconjugates contain one of more modifications in the Fc region that increase the binding of the Fc region of the antibody to FcγRIIB.

In some embodiments, the modulated binding is provided by mutations in the Fc region of the antibody relative to the native Fc region of the antibody. The mutations can be in a CH2 domain, a CH3 domain, or a combination thereof. A “native Fc region” is synonymous with a “wild-type Fc region” and comprises an amino acid sequence that is identical to the amino acid sequence of an Fc region found in nature or identical to the amino acid sequence of the Fc region found in the native antibody (e.g., cetuximab). Native sequence human Fc regions include a native sequence human IgG1 Fc region, native sequence human IgG2 Fc region, native sequence human IgG3 Fc region, and native sequence human IgG4 Fc region, as well as naturally occurring variants thereof. Native sequence Fc includes the various allotypes of Fes (see, e.g., Jefferis et al., mAbs, 1(4): 332-338 (2009)).

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/I332E/A330L), GA (G236A), ALIE (A330L/I332E), GASDALIE (G236A/S239D/A330L/I332E), V9 (G237D/P238D/P271G/A330R), and V11 (G237D/P238D/H268D/P271G/A330R), and/or one or more mutations at the following amino acids: E233, G237, P238, H268, P271, L328 and A330. Additional Fc region modifications for modulating Fc receptor binding are described in, for example, U.S. Patent Application Publication 2016/0145350 and U.S. Pat. Nos. 7,416,726 and 5,624,821, which are hereby incorporated by reference in their entireties.

In some embodiments, the Fc region of the antibodies of the immunoconjugates are modified to have an altered glycosylation pattern of the Fc region compared to the native non-modified Fc region.

Human immunoglobulin is glycosylated at the Asn297 residue in the Cy2 domain of each heavy chain. This V-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose, or sialic acid, which differentially impacts Fc binding to activating and inhibitory FcγR. Additionally, it has been demonstrated that α2,6-sialyation enhances anti-inflammatory activity in vivo, while defucosylation leads to improved FcγRIIIa binding and a 10-fold increase in antibody-dependent cellular cytotoxicity and antibody-dependent phagocytosis. Specific glycosylation patterns, therefore, can be used to control inflammatory effector functions.

In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 and U.S. Patent Application Publications 2007/0014795 and 2008/0286819, which are hereby incorporated by reference in their entireties.

In some embodiments, the antibodies of the immunoconjugates are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies of the immunoconjugates are engineered to be afucosylated.

In some embodiments, the entire Fc region of an antibody in the immunoconjugates is exchanged with a different Fc region, so that the Fab region of the antibody is conjugated to a non-native Fc region. For example, the Fab region of cetuximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1, or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

### Formulation and Administration of Immunoconjugates

In some embodiments, the composition further comprises one or more pharmaceutically acceptable excipients. For example, the immunoconjugates of the invention can be formulated for parenteral administration, such as IV administration or administration into a body cavity or lumen of an organ. Alternatively, the immunoconjugates can be injected intra-tumorally. Formulations for injection will commonly comprise a solution of the immunoconjugate dissolved in a pharmaceutically acceptable carrier. Among the acceptable vehicles and solvents that can be employed are water and an isotonic solution of one or more salts such as sodium chloride, e.g., Ringer's solution. In addition, sterile fixed oils can conventionally be employed as a solvent or suspending medium. For this purpose, any bland fixed oil can be employed, including synthetic monoglycerides or diglycerides. In addition, fatty acids such as oleic acid can likewise be used in the preparation of injectables. These formulations desirably are sterile and generally free of undesirable matter. These formulations can be sterilized by conventional, well known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the immunoconjugate in these formulations can vary widely, and will be selected primarily based on fluid volumes, viscosities, body weight, and the like, in accordance with the particular mode of administration selected and the patient's needs. In certain embodiments, the concentration of an immunoconjugate in a solution formulation for injection will range from about 0.1% (w/w) to about 10% (w/w).

In another aspect, the invention provides a method for treating cancer. The method includes comprising administering a therapeutically effective amount of an immunoconjugate (e.g., as a composition as described above) to a subject in need thereof. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

In a further aspect, the invention provides a method for curing cancer. The method comprises administering a therapeutically effective amount of an immunoconjugate (e.g., as a composition as described above) to a subject. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being cured. Frequency of administration can range from a single dose to multiple doses per week, or more frequently. In some embodiments, the immunoconjugate is administered from about once per month to about five times per week. In some embodiments, the immunoconjugate is administered once per week.

In another aspect, the invention provides a method for preventing cancer. The method comprises administering a therapeutically effective amount of an immunoconjugate (e.g., as a composition as described above) to a subject. In certain embodiments, the subject is susceptible to a certain cancer to be prevented. For example, the methods can include administering the immunoconjugate to provide a dose of from about 100 ng/kg to about 50 mg/kg to the subject. The immunoconjugate dose can range from about 5 mg/kg to about 50 mg/kg, from about 10 μg/kg to about 5 mg/kg, or from about 100 μg/kg to about 1 mg/kg. The immunoconjugate dose can be about 100, 200, 300, 400, or 500 μg/kg. The immunoconjugate dose can be about 1, 2, 3, 4, 5, 6, 7, 8, 9, or 10 mg/kg. The immunoconjugate dose can also be outside of these ranges, depending on the particular conjugate as well as the type and severity of the cancer being treated. Frequency of administration can range from a single dose to multiple doses per week, or more frequently. In some embodiments, the immunoconjugate is administered from about once per month to about five times per week. In some embodiments, the immunoconjugate is administered once per week.

Some embodiments of the invention provide methods for treating cancer as described above, wherein the cancer is a head and neck cancer. Head and neck cancer (as well as head and neck squamous cell carcinoma) refers to a variety of cancers characterized by squamous cell carcinomas of the oral cavity, pharynx and larynx, salivary glands, paranasal sinuses, and nasal cavity, as well as the lymph nodes of the upper part of the neck. Head and neck cancers account for approximately 3 to 5 percent of all cancers in the United States. These cancers are more common in men and in people over age 50. Tobacco (including smokeless tobacco) and alcohol use are the most important risk factors for head and neck cancers, particularly those of the oral cavity, oropharynx, hypopharynx and larynx. Eighty-five percent of head and neck cancers are linked to tobacco use.

In the methods of the invention, the immunoconjugates can be used to target a number of malignant cells. For example, the immunoconjugates can be used to target squamous epithelial cells of the lip, oral cavity, pharynx, larynx, nasal cavity, or paranasal sinuses. The immunoconjugates can be used to target mucoepidermoid carcinoma cells, adenoid cystic carcinoma cells, adenocarcinoma cells, small-cell undifferentiated cancer cells, esthesioneuroblastoma cells, Hodgkin lymphoma cells, and Non-Hodgkin lymphoma cells.

Some embodiments of the invention provide methods for treating cancer as described above, wherein the cancer is breast cancer. Breast cancer can originate from different areas in the breast, and a number of different types of breast cancer have been characterized. For example, the immunoconjugates of the invention can be used for treating ductal carcinoma in situ; invasive ductal carcinoma (e.g., tubular carcinoma; medullary carcinoma; mucinous carcinoma; papillary carcinoma; or cribriform carcinoma of the breast); lobular carcinoma in situ; invasive lobular carcinoma; inflammatory breast cancer; and other forms of breast cancer. In some embodiments, methods for treating breast cancer include administering an immunoconjugate containing an antibody that is capable of binding EGFR (e.g., cetuximab).

In some embodiments, the cancer is susceptible to a pro-inflammatory response induced by TLR7 and/or TLR8.

Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-32 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

1. An Immunoconjugate of Formula:

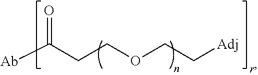

or pharmaceutically acceptable salt thereof, wherein subscript r is an integer from 1 to 10, subscript n is an integer from about 2 to about 50, “Adj” is an adjuvant moiety, and “Ab” is an antibody construct that has an antigen binding domain that binds EGFR.

2. The immunoconjugate of aspect 1, wherein the adjuvant moiety is a TLR7 and/or TLR8 agonist.

3. The immunoconjugate of aspect 1 or 2, wherein the adjuvant moiety is of formula:

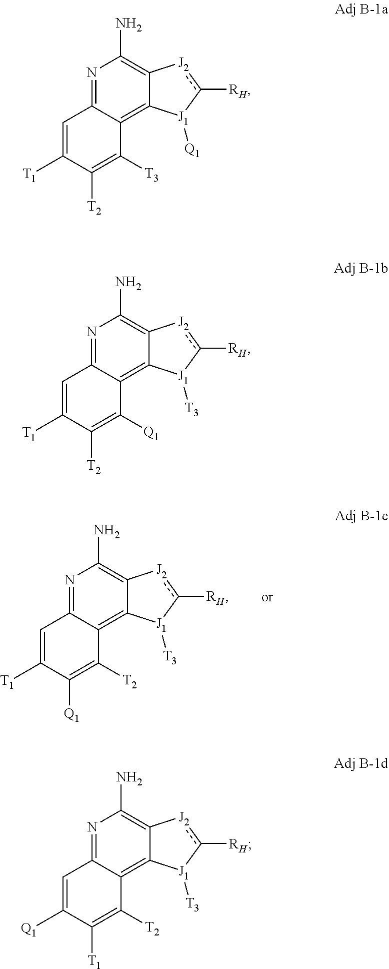

wherein

J₁ is CH or N,

J₂ is CH, CH₂, N, NH, O, or S,

Q₁ is of the formula:

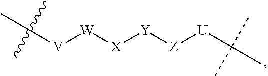

T₁, T₂, T₃, and R_{H} independently are of the formula:

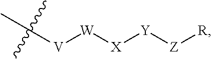

each V is optionally present and independently is —O—, —S—, —NH—, —NR—, or —CO—,

each W is optionally present and independently is a linear or branched, saturated or unsaturated, divalent C₁-C₈ alkyl,

each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

each Y is optionally present and independently is —CO— or a linear or branched, saturated or unsaturated, divalent C₁-C₈ alkyl,

each Z is optionally present and independently is —O—, —S—, —NH—, or —NR—,

U is optionally present and is

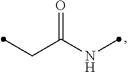

each R independently is hydrogen, halogen (e.g., fluorine, chlorine, bromine, or iodine), nitrile, —COOH, or a linear or branched, saturated or unsaturated C₁-C₄ alkyl,

“ [symbol] ” represents a single bond or a double bond,

the wavy line (“ [symbol] ”) represents a point of attachment of Q₁, T₁, T₂, T₃, and R_{H},

the dot (“●”) represents a point of attachment of U, and

the dashed line (“ [symbol] ”) represents a point of attachment of the adjuvant moiety.

4. The immunoconjugate of aspect 3, wherein the adjuvant moiety is of formula:

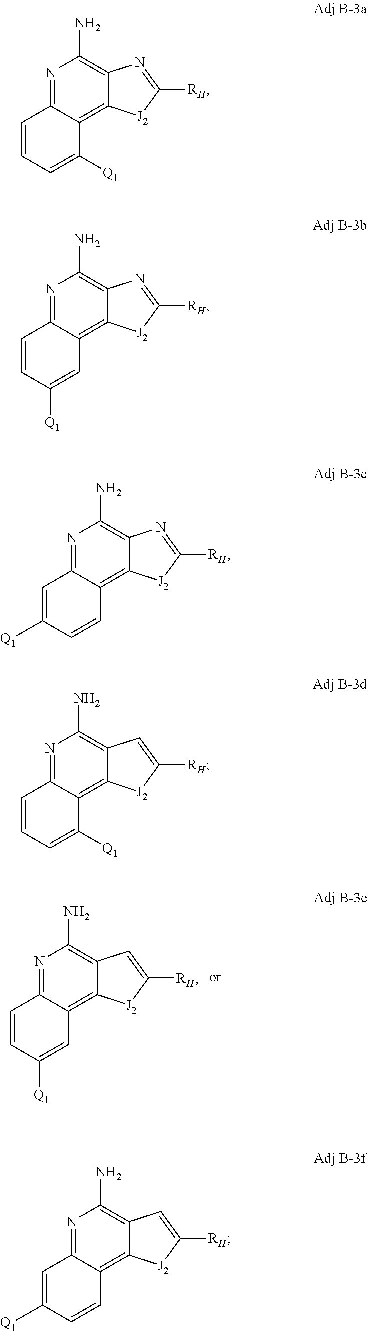

wherein

J₂ is CH₂, NH, O, or S,

Q₁ is of the formula:

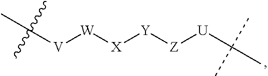

R_{H} is of the formula:

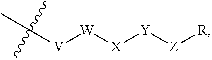

each V is optionally present and independently is —O—, —S—, —NH—, —NR—, or —CO—,

each W is optionally present and independently is a linear or branched, saturated or unsaturated, divalent C₁-C₈ alkyl,

each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

each Y is optionally present and independently is —CO— or a linear or branched, saturated or unsaturated, divalent C₁-C₈ alkyl,

each Z is optionally present and independently is —O—, —S—, —NH—, or —NR—,

U is optionally present and is

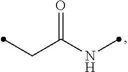

each R independently is hydrogen, halogen (e.g., fluorine, chlorine, bromine, or iodine), nitrile, —COOH, or a linear or branched, saturated or unsaturated C₁-C₄ alkyl,

the wavy line (“ [symbol] ”) represents a point of attachment of Q₁ and R_{H},

the dot (“●”) represents a point of attachment of U, and

the dashed line (“ [symbol] ”) represents a point of attachment of the adjuvant moiety.

5. The immunoconjugate of aspect 4, wherein the adjuvant moiety is of formula:

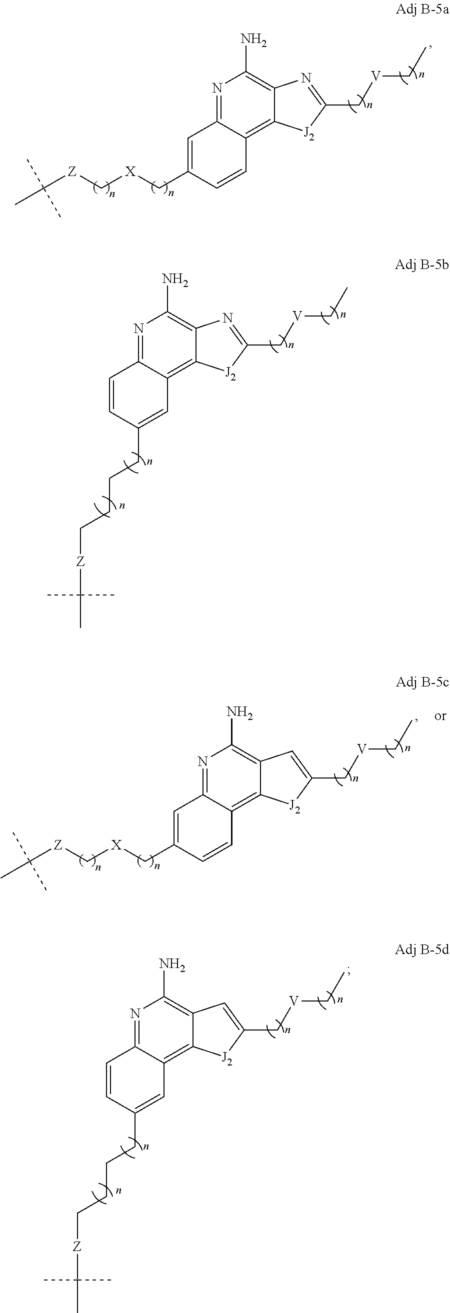

wherein

J₂ is CH₂, NH, O, or S,

V is optionally present and is —O—, —S—, —NH—, —NR—, or —CO—,

X is optionally present and is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,

Z is optionally present and is —O—, —S—, —NH—, or —NR—,

provided that at least X or Z is present,

each R independently is hydrogen, halogen (e.g., fluorine, chlorine, bromine, or iodine), nitrile, —COOH, or a linear or branched, saturated or unsaturated C₁-C₄ alkyl,

each n independently is an integer from 0 to 4, and

the dashed line (“ [symbol] ”) represents a point of attachment of the adjuvant moiety.

6. The immunoconjugate of aspect 5, wherein the adjuvant moiety is of formula:

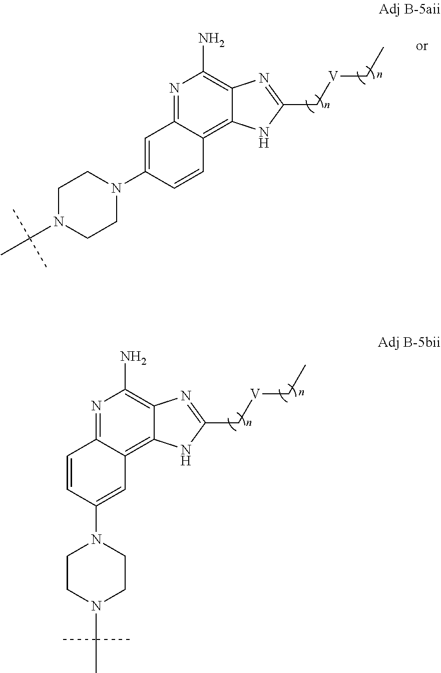

wherein

V is optionally present and is —O—, —S—, —NH—, —NR—, or —CO—,

R is hydrogen, halogen (e.g., fluorine, chlorine, bromine, or iodine), nitrile, —COOH, or a linear or branched, saturated or unsaturated C₁-C₄ alkyl,

each n independently is an integer from 0 to 4, and

the dashed line (“ [symbol] ”) represents a point of attachment of the adjuvant moiety.

7. The immunoconjugate of aspect 1, wherein the immunoconjugate is of formula:

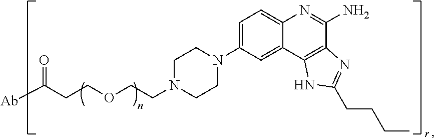

or pharmaceutically acceptable salt thereof, wherein subscript r is an integer from 1 to 10, subscript n is an integer from about 2 to about 50, and “Ab” is an antibody construct that has an antigen binding domain that binds EGFR.

8. The immunoconjugate of any one of aspects 1-7, wherein subscript r is an integer from 1 to 6.

9. The immunoconjugate of aspect 8, wherein subscript r is an integer from 1 to 4.

10. The immunoconjugate of aspect 9, wherein subscript r is 1.

11. The immunoconjugate of aspect 9, wherein subscript r is 2.

12. The immunoconjugate of aspect 9, wherein subscript r is 3.

13. The immunoconjugate of aspect 9, wherein subscript r is 4.

14. The immunoconjugate of any one of aspects 1-13, wherein subscript n is an integer from 2 to 25.

15. The immunoconjugate of aspect 14, wherein subscript n is an integer from 6 to 25.

16. The immunoconjugate of aspect 15, wherein subscript n is an integer from 8 to 16.

17. The immunoconjugate of aspect 16, wherein subscript n is an integer from 8 to 12.

18. The immunoconjugate of aspect 7, wherein the immunoconjugate is of formula:

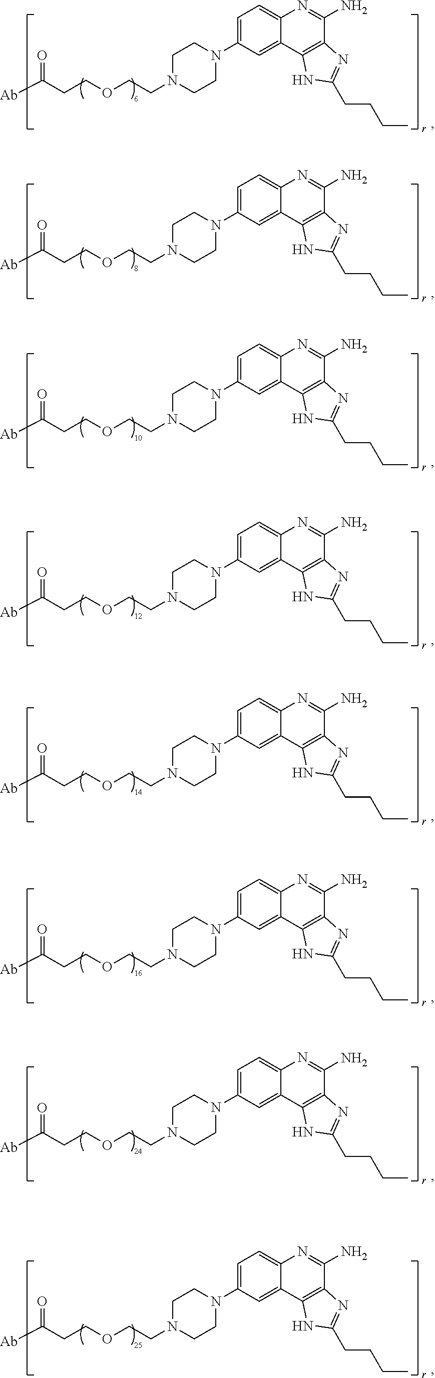

or pharmaceutically acceptable salt thereof, wherein subscript r is an integer from 1 to 10 and “Ab” is an antibody construct that has an antigen binding domain that binds EGFR.

19. The immunoconjugate of any one of aspects 1-18, wherein “Ab” is cetuximab, panitumumab, or necitumumab, a biosimilar thereof, or a biobetter thereof.

20. The immunoconjugate of aspect 19, wherein “Ab” is cetuximab.

21. The immunoconjugate of aspect 19, wherein “Ab” is panitumumab.

22. The immunoconjugate of aspect 19, wherein “Ab” is necitumumab.

23. The immunoconjugate of aspect 19, wherein “Ab” is STI-001, RPH-002, CMAB009, ONS-1055, MabionEGFR, HLX-05, HLX05, CT-P15, KN-005, ABP-494, AP 087, tomuzotuximab, GC1118, SYN004, SCT200, orHLX-07.

24. A composition comprising a plurality of immunoconjugates according to any one of aspects 1-23.

25. The composition of aspect 24, wherein the average drug to antibody ratio is from about 0.01 to about 10.

26. The composition of aspect 25, wherein the average drug to antibody ratio is from about 1 to about 10.

27. The composition of aspect 26, wherein the average drug to antibody ratio is from about 1 to about 6.

28. The composition of aspect 27, wherein the average drug to antibody ratio is from about 1 to about 4.

29. The composition of aspect 28, wherein the average drug to antibody ratio is from about 1 to about 3.

30. A therapeutically effective amount of an immunoconjugate according to any one of aspects 1-23 or a composition according to any one of aspects 24-29 for use in a method of treating cancer.

31. An immunoconjugate according to any one of aspects 1-23 or a composition according to any one of aspects 24-29 for use in a method of treating cancer.

32. The immunoconjugate according to any one of aspects 1-23 or a composition according to any one of aspects 24-29 for the use of aspect 30 or 31, wherein the cancer is susceptible to a pro-inflammatory response induced by TLR7 and/or TLR8 agonism.

### EXAMPLES

The following example further illustrates the invention but, of course, should not be construed as in any way limiting its scope.

### Example 1: Treatment of Colorectal Cancer with an Immunoconjugate of the Invention

This example demonstrates the ability of the immunoconjugates of the invention to act as potent anti-tumor therapies, as exhibited by treatment of a human tumor model for colorectal cancer, COLO 205.

This example employed a humanized mouse model, in which immunodeficient mice are simultaneously engrafted with human peripheral blood mononuclear cells (“PBMCs”) and a human tumor xenograft. PBMCs from healthy donors were isolated and depleted of NK cells. A human tumor model for colorectal cancer, COLO 205, was utilized, as this tumor is known to highly express the tumor antigen EGFR, enabling the use of the clinical monoclonal antibody cetuximab. The COLO 205 tumor cells were prepared as follows.

The COLO 205 tumor cells were maintained in vitro in RPMI-1640 medium supplemented with 10% fetal bovine serum at 37° C. in an atmosphere of 5% CO₂ in air. The cells in exponential growth phase were harvested and quantitated by cell counter before tumor inoculation.

PBMCs were isolated from blood of two healthy donors by density gradient centrifugation using standard procedures. After centrifugation, cells were washed with phosphate buffered saline (“PBS”) solution and resuspended in PBS. PBMCs will be depleted in NK cells, using CD56 microbeads (Miltenyi or similar) for administration. In order to ensure the highest deletion in NK cells, 2 rounds of purification were performed. Cell number was adjusted to 8×10⁷×(1−NK %) cells/ml (4×10⁶×(1−NK %)/50 ul) for inoculation.

Each mouse was inoculated subcutaneously at the right flank region with COLO 205 tumor cells (4×10⁶) in 0.05 ml of PBS admixed with PBMCs-depleted in NK (4×10⁶×(1−NK %)) in 0.05 ml of PBS for tumor development.

Engrafted mice were then treated systemically either with cetuximab or Immunoconjugate 1.

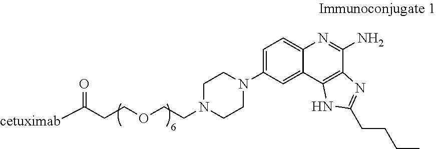

To determine the DAR, Immunoconjugate 1 was acidified (diluted 5 fold or more in water, 0.2% formic acid) and injected onto a Waters BEH-C4 reverse phase column (product number 186004495) hooked up to a Waters Aquity H-class UPLC and separated using a linear gradient of 1-90% acetonitrile, 0.1% formic acid. C4 column eluates are continuously analyzed via electrospray ionization onto a Waters Xevo G2-XS time of flight (TOF) mass spectrometer. To determine the DAR for a conjugate, it is first necessary to identify the time window in the total ion current chromatogram (TIC) that corresponds to the elution window for the antibody conjugate from the C4 column. Once selected, the observed ions, representing several co-eluting families of mass/charge (m/z) species (one family for each protein species) within the given time window are deconvoluted using Water's MassLynx v4.1 software into accurate masses for each DAR species present. The intensity of the peaks for each DAR species is then combined using equation 1:

$$Average DAR = \frac{\begin{matrix}( 1 \times iDAR1 ) + ( 2 \times iDAR2 ) + \\ ( 3 \times iDAR3 ) + ( 4 \times iDAR4 )\end{matrix}}{\begin{matrix}iDAR 0 + i D AR1 + \\ iDAR2 + i D A R 3 + i D AR4\end{matrix}} \qquad Eq . 1$$

wherein iDAR is equal to the observed peak intensity (observed ions) for a given DAR species and the total number of observed species is five (four DAR species+unlabeled antibody). The equation may be adjusted as required for the number of species present. This equation is for an antibody conjugate that has been deglycosylated prior to LC-MS analysis. For analysis of a glycosylated antibody each DAR species may be represented by multiple peaks within the deconvoluted time window. In this case iDARn=[n×(iDARn_{gly1}+iDARn_{gly2}+iDARn_{gly3})] where n is the DAR species and the number of observed glycosylation variants is three for example.

Immunoconjugate 1 with cetuximab as the antibody had a DAR of 2.2, as analyzed using the adjuvant activity and immunoconjugate activity procedures described herein.

Treatment started 4 days after tumor cell inoculation when the mean tumor volume was around 50-80 mm³. The date of tumor cell inoculation was denoted as day 0. Before commencement of treatment, all animals were weighed. All animals were randomly allocated to 4 study groups. Randomization was performed based on “Matched distribution” method (STUDYDIRECTOR™ software, version 3.1.399.19).

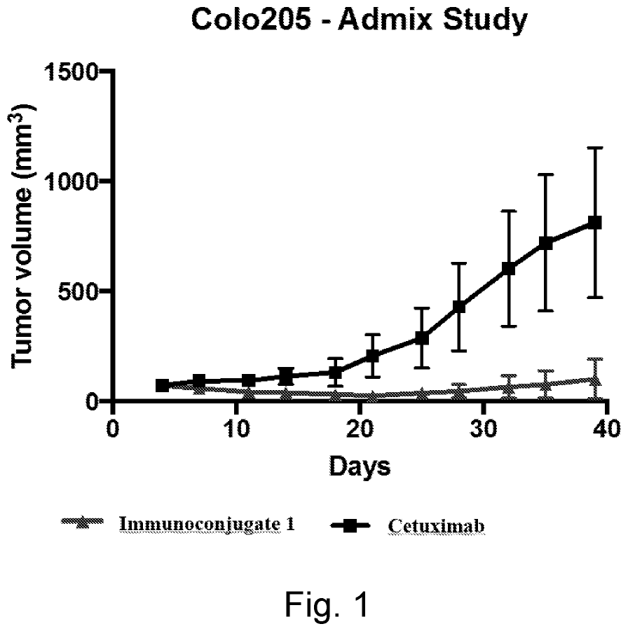

Tumor volumes were measured twice per week after randomization in two dimensions using a caliper, and the volume was expressed in mm³ using the formula: “V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were calculated using STUDYDIRECTOR™ software (version 3.1.399.19). The results are set forth in FIG. 1.

FIG. 1 shows that the antibody alone was not sufficient for control of tumor growth. In contrast, treatment with Immunoconjugate 1 led to robust anti-tumor effects as demonstrated the significant reduction in size relative to treatment with cetuximab. In addition, treatment with Immunoconjugate 1 was successful in curing four of seven mice of cancer.

### Example 2: Treatment of Lung Adenocarcinoma with an Immunoconjugate of the Invention

This example demonstrates the ability of the immunoconjugates of the invention to act as potent anti-tumor therapies, as exhibited by treatment of a human xenograft tumor model for lung adenocarcinoma, HCC827.

The HCC827 tumor cell line was purchased from American Type Culture Collection (ATCC™; Manassas, Virgina) and grown according to the manufacturer's guidelines. Cells were harvested when they reached 80-90% confluency by detaching with ACCUTASE™ (Stemcell), washed with PBS, resuspended at 40×10⁶ cells/mL in PBS, and placed on ice for no longer than two hours. Immediately prior to implantation, suspended cells were mixed with an equal volume of CULTREX™ PathClear BME, Type 3 (R&D Systems), and 100 μL of the mixture (2×10⁶ cells) were implanted subcutaneously into the right flank of 6-8-week-old female Rag2/IL2rg double knockout mice (Taconic).

Tumor size was recorded twice a week and was estimated using the following formula: (length×width²)/2. Once tumors reached about 120 mm³, treatments were initiated. Engrafted mice were then treated systemically either with cetuximab or Immunoconjugate 2.

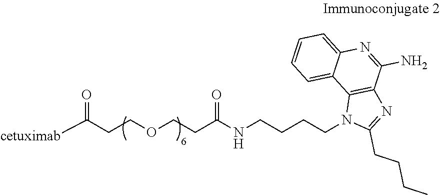

Immunoconjugate 2 with cetuximab as the antibody had a DAR of 2.4, as analyzed using the adjuvant activity and immunoconjugate activity procedures described herein.

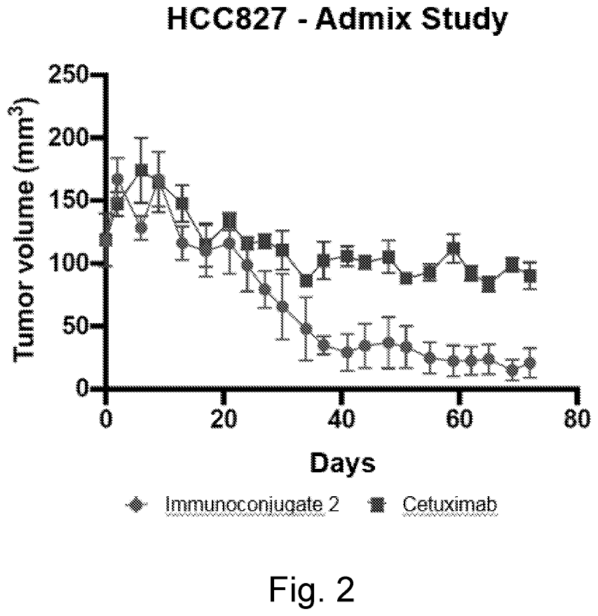

Each of Immunoconjugate 2 and cetuximab was prepared in PBS and administered at 1 mg/kg intraperitoneally twice weekly (BIW×6) for a total of six doses. The results are set forth in the FIG. 2.

FIG. 2 shows that treatment with Immunoconjugate 2 led to robust anti-tumor effects as demonstrated the significant reduction in size relative to treatment with cetuximab.

### Example 3. Assessment of Immunoconjugate Activity In Vitro

This example shows that Immunoconjugates 1 and 2 are effective at eliciting myeloid activation, and therefore are useful for the treatment of cancer.

Isolation of Human Antigen Presenting Cells. Human myeloid antigen presenting cells (APCs) were negatively selected from human peripheral blood obtained from healthy blood donors (Stanford Blood Center, Palo Alto, Calif.) by density gradient centrifugation using a ROSETTESEP™ Human Monocyte Enrichment Cocktail (Stem Cell Technologies, Vancouver, Canada) containing monoclonal antibodies against CD14, CD16, CD40, CD86, CD123, and HLA-DR. Immature APCs were subsequently purified to >97% purity via negative selection using an EASYSEP™ Human Monocyte Enrichment Kit (Stem Cell Technologies) without CD16 depletion containing monoclonal antibodies against CD14, CD16, CD40, CD86, CD123, and HLA-DR.

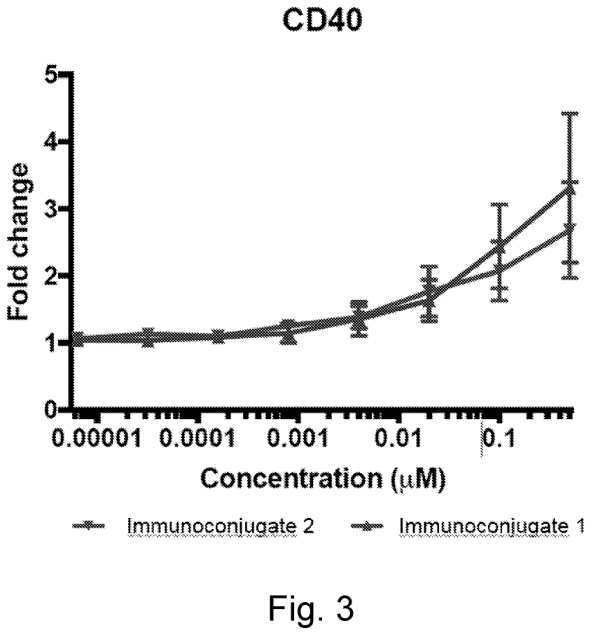

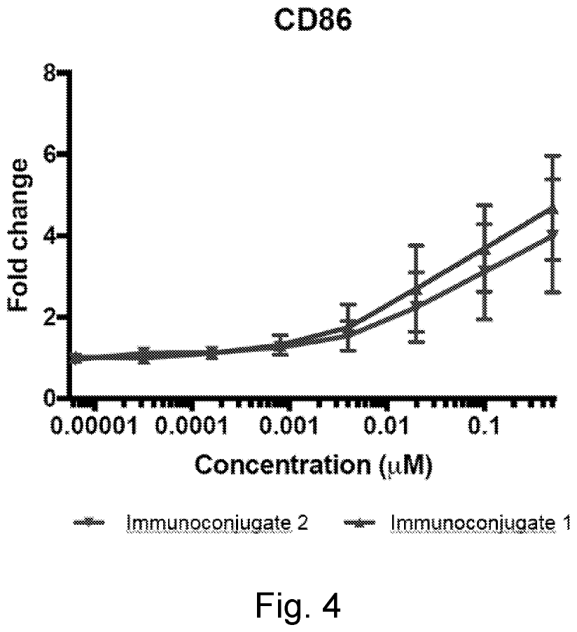

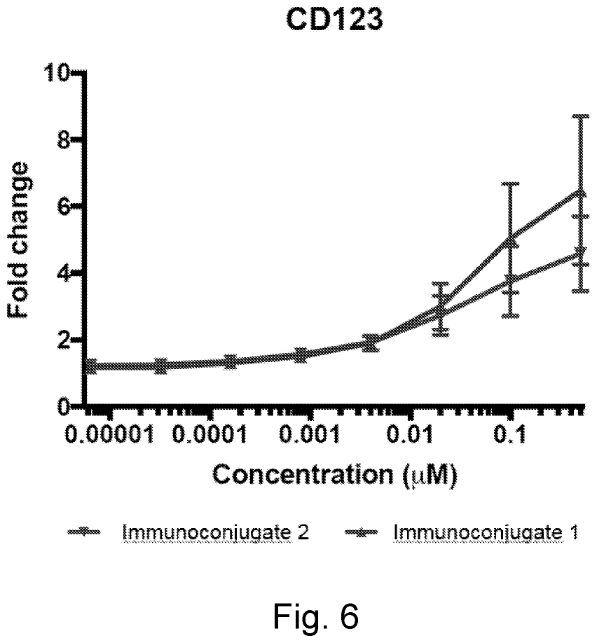

APC Activation. 2×10⁵ APCs were incubated in 96-well plates (Corning, Corning, N.Y.) containing iscove's modified dulbecco's medium (IMDM) (Thermo Fisher Scientific) supplemented with 10% FBS, 100 U/mL penicillin, 100 μg/mL streptomycin, 2 mM L-glutamine, sodium pyruvate, non-essential amino acids, and, where indicated, various concentrations of Immunoconjugate 1 and Immunoconjugate 2 of the invention. Cells were analyzed after 18 hours via flow cytometry. The results of this assay are shown in the FIGS. 3-6.

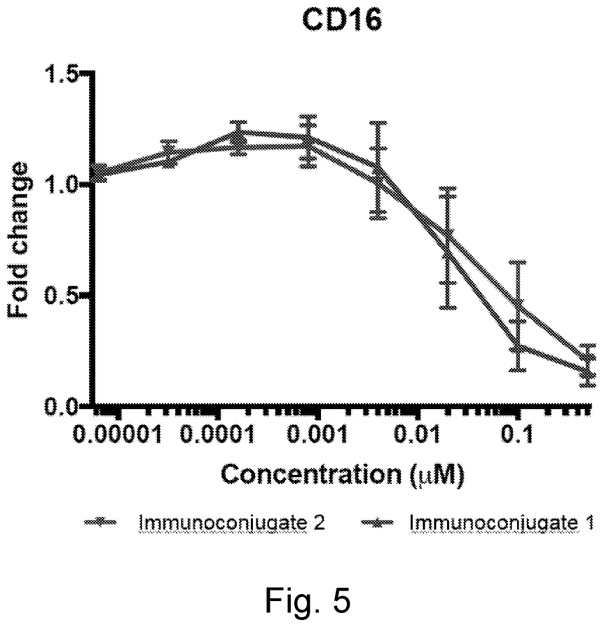

As is apparent from FIGS. 3, 4, and 6, Immunoconjugates 1 and 2 elicit myeloid activation as indicated by CD40, CD86, and CD123 upregulation, respectively. FIG. 5 demonstrates that Immunoconjugates 1 and 2 elicit myeloid differentiation as indicated by CD16 downregulation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An immunoconjugate of formula: 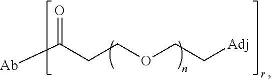
or pharmaceutically acceptable salt thereof, wherein subscript r is an integer from 1 to 10, subscript n is an integer from about 2 to about 50, “Adj” is an adjuvant moiety, and “Ab” is an antibody construct that has an antigen binding domain that binds EGFR.

2. The immunoconjugate of claim 1, wherein the adjuvant moiety is a TLR7 and/or TLR8 agonist.

3. The immunoconjugate of claim 1, wherein the adjuvant moiety is of formula: 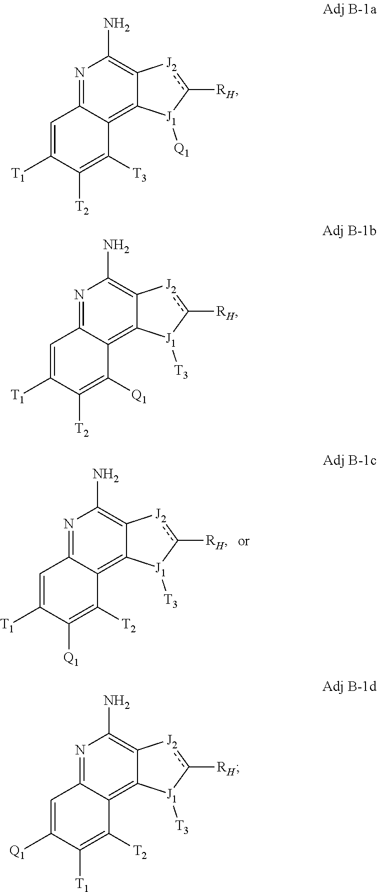
wherein
J₁ is CH or N,
J₂ is CH, CH₂, N, NH, O, or S,
Q₁ is of the formula: 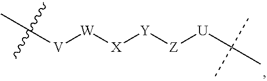
T₁, T₂, T₃, and R_{H} independently are of the formula: 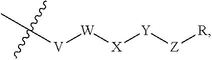
each V is optionally present and independently is —O—, —S—, —NH—, —NR—, or —CO—,
each W is optionally present and independently is a linear or branched, saturated or unsaturated, divalent C₁-C₈ alkyl,
each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,
each Y is optionally present and independently is —CO— or a linear or branched, saturated or unsaturated, divalent C₁-C₈ alkyl,
each Z is optionally present and independently is —O—, —S—, —NH—, or —NR—,
U is optionally present and is 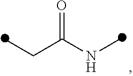
each R independently is hydrogen, halogen (e.g., fluorine, chlorine, bromine, or iodine), nitrile, —COOH, or a linear or branched, saturated or unsaturated C₁-C₄ alkyl,
“ [symbol] ” represents a single bond or a double bond,
the wavy line (“ [symbol] ”) represents a point of attachment of Q₁, T₁, T₂, T₃, and R_{H},
the dot (“●”) represents a point of attachment of U, and
the dashed line (“ [symbol] ”) represents a point of attachment of the adjuvant moiety.

4. The immunoconjugate of claim 3, wherein the adjuvant moiety is of formula: 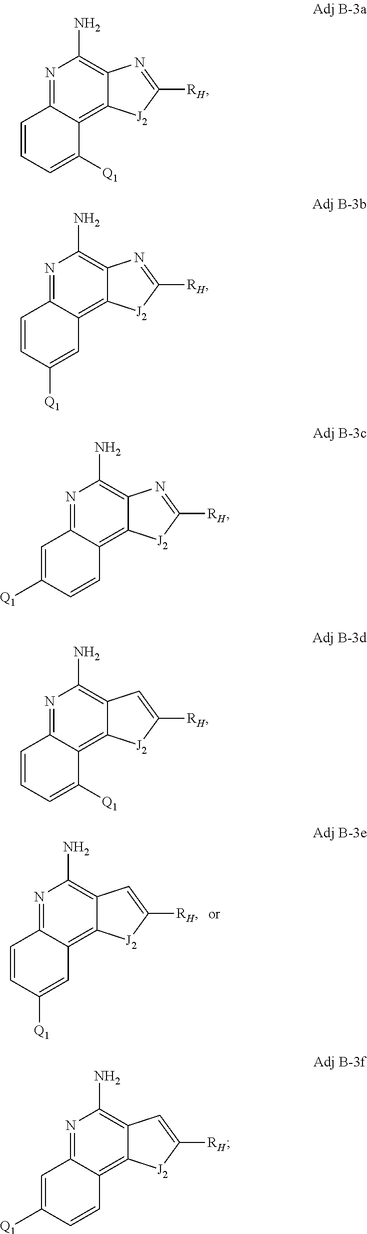
wherein
J₂ is CH₂, NH, 0, or S,
Q₁ is of the formula: 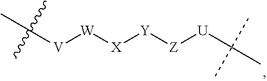
R_{H} is of the formula: 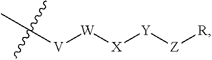
each V is optionally present and independently is —O—, —S—, —NH—, —NR—, or —CO—,
each W is optionally present and independently is a linear or branched, saturated or unsaturated, divalent C₁-C₈ alkyl,
each X is optionally present and independently is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,
each Y is optionally present and independently is —CO— or a linear or branched, saturated or unsaturated, divalent C₁-C₈ alkyl,
each Z is optionally present and independently is —O—, —S—, —NH—, or —NR—,
U is optionally present and is 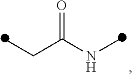
each R independently is hydrogen, halogen (e.g., fluorine, chlorine, bromine, or iodine), nitrile, —COOH, or a linear or branched, saturated or unsaturated C₁-C₄ alkyl,
the wavy line (“ [symbol] ”) represents a point of attachment of Q₁ and R_{H},
the dot (“●”) represents a point of attachment of U, and
the dashed line (“ [symbol] ”) represents a point of attachment of the adjuvant moiety.

5. The immunoconjugate of claim 4, wherein the adjuvant moiety is of formula: 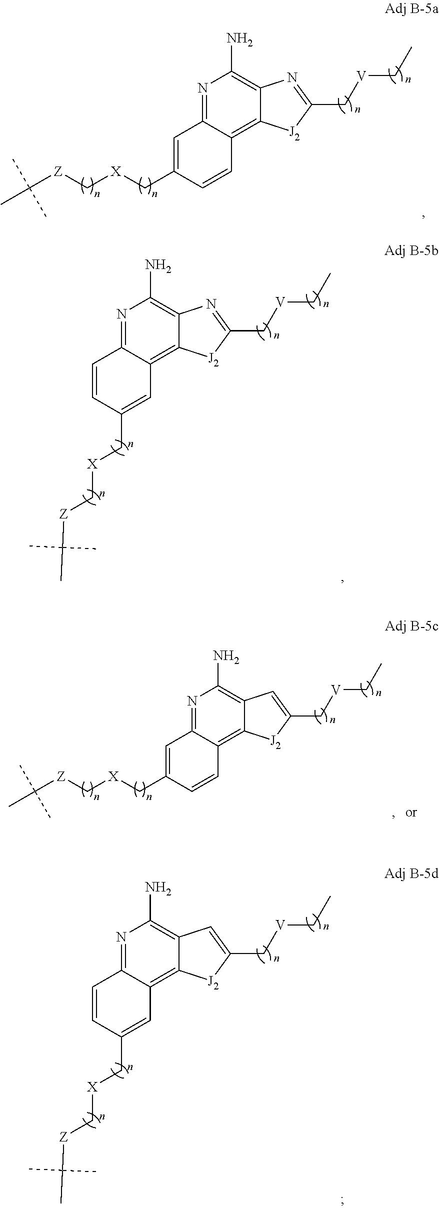
wherein
J₂ is CH2, NH, O, or S,
V is optionally present and is —O—, —S—, —NH—, —NR—, or —CO—,
X is optionally present and is one, two, three, or four divalent cycloalkyl, heterocycloalkyl, aryl, or heteroaryl groups, and when more than one divalent cycloalkyl, heterocycloalkyl, aryl, or heteroaryl group is present, the more than one divalent cycloalkyl, heterocycloalkyl, aryl, or heteroaryl groups are linked or fused, wherein linked divalent cycloalkyl, heterocycloalkyl, aryl, or heteroaryl groups are linked through a bond or —CO—,
Z is optionally present and is —O—, —S—, —NH—, or —NR—,
provided that at least X or Z is present,
each R independently is hydrogen, halogen (e.g., fluorine, chlorine, bromine, or iodine), nitrile, —COOH, or a linear or branched, saturated or unsaturated C₁-C₄ alkyl,
each n independently is an integer from 0 to 4, and
the dashed line (“ [symbol] ”) represents a point of attachment of the adjuvant moiety.

6. The immunoconjugate of claim 5, wherein the adjuvant moiety is of formula: 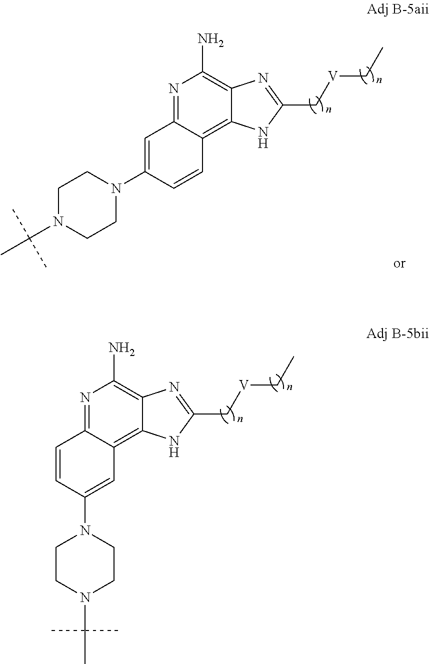
wherein
V is optionally present and is —O—, —S—, —NH—, —NR—, or —CO—,
R is hydrogen, halogen (e.g., fluorine, chlorine, bromine, or iodine), nitrile, —COOH, or a linear or branched, saturated or unsaturated C₁-C₄ alkyl,
each n independently is an integer from 0 to 4, and
the dashed line (“ [symbol] ”) represents a point of attachment of the adjuvant moiety.

7. The immunoconjugate of claim 1, wherein the immunoconjugate is of formula: 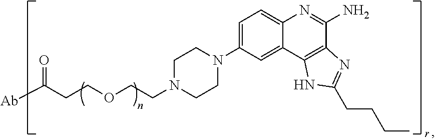
or pharmaceutically acceptable salt thereof, wherein subscript r is an integer from 1 to 10, subscript n is an integer from about 2 to about 50, and “Ab” is an antibody construct that has an antigen binding domain that binds EGFR.

8. The immunoconjugate of claim 1, wherein subscript r is an integer from 1 to 6.

9. The immunoconjugate of claim 8, wherein subscript r is an integer from 1 to 4.

10.-15. (canceled)

16. The immunoconjugate of claim 1, wherein subscript n is an integer from 8 to 16.

17. (canceled)

18. The immunoconjugate of claim 7, wherein the immunoconjugate is of formula: 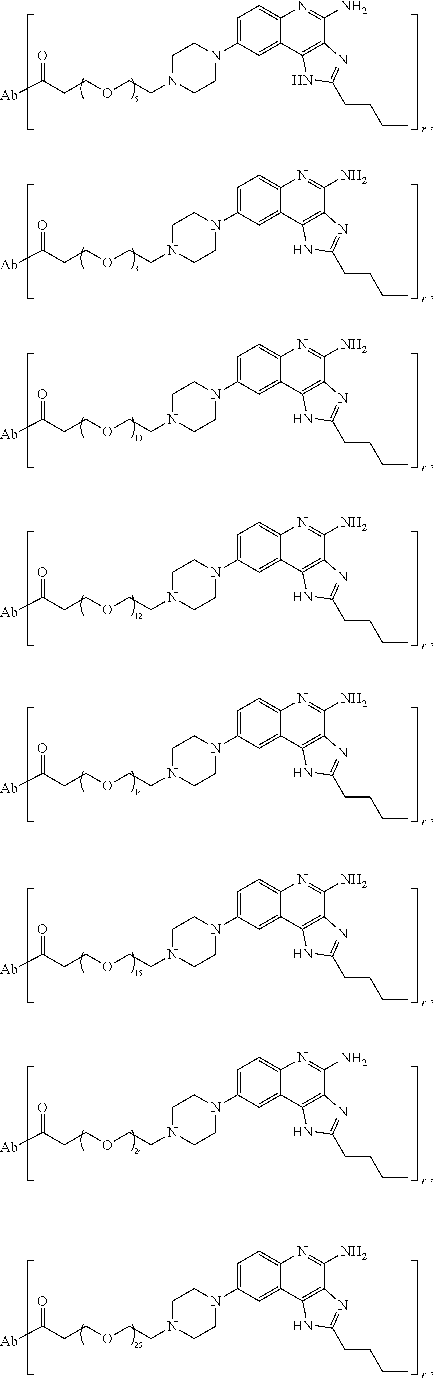
or pharmaceutically acceptable salt thereof, wherein subscript r is an integer from 1 to 10 and “Ab” is an antibody construct that has an antigen binding domain that binds EGFR.

19. The immunoconjugate of claim 1, wherein “Ab” is cetuximab, panitumumab, or necitumumab, a biosimilar thereof, or a biobetter thereof.

20.-22. (canceled)

23. The immunoconjugate of claim 19, wherein “Ab” is STI-001, RPH-002, CMAB009, ONS-1055, MabionEGFR, HLX-05, HLX05, CT-P15, KN-005, ABP-494, AP-087, tomuzotuximab, GC1118, SYN004, SCT200, or HLX-07.

24. A composition comprising a plurality of immunoconjugates according to claim 1.

25. The composition of claim 24, wherein the average drug to antibody ratio is from about 0.01 to about 10.

26. The composition of claim 25, wherein the average drug to antibody ratio is from about 1 to about 10.

27.-32. (canceled)

33. A method for treating cancer comprising administering a therapeutically effective amount of an immunoconjugate according to claim 1 to a subject in need thereof.

34. The method of claim 33, wherein the cancer is susceptible to a pro-inflammatory response induced by TLR7 and/or TLR8 agonism.

35. A method for treating cancer comprising administering a therapeutically effective a composition according to claim 24 to a subject in need thereof.

36. The method of claim 35, wherein the cancer is susceptible to a pro-inflammatory response induced by TLR7 and/or TLR8 agonism.
